(19) United States
(12) Patent Application Publication
(10) Pub. No.: US 2025/0127697 A1
(43) Pub. Date: Apr. 24, 2025

(54) HAIR TREATMENT COMPOSITION WITH CATIONIC SURFACTANT, FATTY ALCOHOLS, AND LOW POLAR OIL

(71) Applicant: L'OREAL, Paris (FR)

(72) Inventors: Jun LIANG, Brooklyn, NY (US); Vibha SHAH, Branchburg, NJ (US)

(21) Appl. No.: 18/490,618

(22) Filed: Oct. 19, 2023

(51) Int. Cl. A61K 8/41; A61K 8/06; A61K 8/34; A61Q 5/06; A61Q 5/12

(52) U.S. Cl. CPC A61K 8/416; A61K 8/062; A61K 8/342; A61K 8/345; A61Q 5/06; A61Q 5/12; A61K 2800/5922

(57) ABSTRACT

Hair treatment compositions and methods for treating hair using the compositions are disclosed. The hair treatment compositions include: (a) one or more cationic surfactants; (b) one or more solid fatty alcohols; (c) one or more liquid fatty alcohols; (d) one or more low polar oils having a Polarity Index of about 23 mN/m to about 40 mN/m; and (e) water; wherein a weight ratio of a combination of (c) and (d) to a combination of (a) and (b) is from about 0.5:1 to about 3:1 (((c)+(d)):((a)+(b))). The hair treatment compositions are particularly useful in methods for detangling, aligning, and smoothing the hair.

### FIELD OF THE DISCLOSURE

The present disclosure relates to hair treatment compositions and methods for treating hair with the hair treatment compositions. The hair treatment compositions and methods are useful for detangling, aligning, and smoothing hair.

### BACKGROUND

Many consumers desire to use cosmetic and care compositions that enhance the appearance of keratinous substrates such as hair, e.g., by changing the color, style, and/or shape of the hair, and/or by imparting various cosmetic properties to hair, such as shine and conditioning. Hair can become dry or damaged for various reasons, e.g., weather exposure, poor nutrition, mechanical treatments (e.g. brushing hair), styling treatments using chemicals, dying, heat, nutrition, etc. Even cleansing products can remove hair's natural oils causing dryness, which can lead to a dull appearance and to split ends. Individuals with curly hair can also encounter additional challenges when caring for their hair. Curly hair more easily tangles and can be more difficult to manage.

Environmental factors, such as salt water, sunlight, and heat, and chemical treatments are known to damage hair. Various types of chemical treatments include, for example, hair bleaching and coloring, permanents, waving products, and relaxing treatments (straightening treatments). These chemical treatments change the look of hair by changing its physical structure, which inevitably causes a certain degree of damage to the hair.

Damaged hair is characterized by unnatural changes to the protein structure of the individual hair strands or shafts. Damage results in split ends, dry straw-like hair, hair that is easily broken, and hair that is “frizzy” and unmanageable. Because the visible portion of hair is dead, it has no ability to regenerate itself.

There are numerous over the counter and salon treatments that purport to repair damaged hair. These include conditioners, hot oil treatments, hydrolyzed proteins, vitamin formulations, and exotic fruit, leaf, or root extracts. These treatments, however, provide only limited improvement to the hair. Therefore, hair repair technologies that restore the properties of hair back to their natural level are desired.

Silicones have been used to add lubricity and shine to hair. Silicones work by coating the cuticle, the outermost layer of the hair fiber. Silicones are often found in conditioners because they increase slip, help lock-in moisture, and leave the hair feeling soft. The increased “slip” helps ease detangling of the hair. Silicones are also used for their anti-frizz properties because they can form a coating on the hair that resists humidity that causes frizz. However, silicones can build-up on the hair leaving a residue that is difficult to wash out. The residue ultimately prevents moisture from absorbing into the hair, thereby actually causing dryness and damage over time. In addition, silicones can weigh down the hair, especially fine hair, causing it to lose volume and bounce.

The popularity and usage of oils for dry hair treatments has increased due to their effectiveness and simplicity. Commonly used oils include olive oil, mineral oil, avocado oil, apricot kernel oil, rice bran oil, and coconut oil. However, these treatments can leave the hair feeling greasy. In addition, the effects are not usually seen after more than several hours (e.g. 8 hours) of treatment, and several treatments are usually required, making it time consuming and labor intensive. Other fatty compounds, such as waxes and other high melting point fatty compounds, have also been incorporated into hair treatment compositions to add lubricity and smoothness to the hair. Such products, however, tend cause the hair to feel and look “greasy” and worse, upon drying, they can form a waxy, whitish, residue on the hair fibers, which is visually perceptible. The whitish residue tends to break apart from the hair, causing flaking. Flaking is particularly objectionable to consumers because it looks like dandruff. The hair loses its shine, has a flat, waxy appearance with flaky particles, and appears unkept and in need of cleansing.

There is still a need for providing improved manageability to hair, especially curly hair, which is more likely to suffer from tangling. Thus, there is a need for compositions that ease and expedite the detangling of hair, especially curly hair, improve fiber alignment, reduce frizz, and increase shine.

### SUMMARY OF THE DISCLOSURE

The instant disclosure is drawn to hair treatment compositions and methods for treating the hair using the hair treatment compositions. The compositions include a unique combination of cationic surfactants, solid and liquid fatty alcohols, and low polar oils, incorporated into an aqueous medium for delivery to the hair. The compositions are stable, accelerate and ease detangling of hair, and impart a pleasant smoothness and shine to the hair. Despite the use of various non-silicone fatty compounds, the compositions do not form a whitish residue upon drying and therefore do not cause flaking, nor do the compositions leave the hair sticky or greasy. Instead, the hair treated with the composition enjoys a naturally looking shine, is moisturized/hydrated, and feels soft.

The hair treatment compositions typically include:
(a) one or more cationic surfactants;
(b) one or more solid fatty alcohols;
(c) more liquid fatty alcohols;
(d) one or more low polar oils having a Polarity Index of about 23 mN/m to about 40 mN/m; and
(e) water;
   wherein a weight ratio of a combination of (c) and (d) to a combination of (a) and (b) is from about 0.5:1 to about 3:1 (((c)+(d)):((a)+(b))).

Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, and mixtures thereof.

Nonlimiting examples of solid fatty alcohol include stearyl alcohol, cetyl alcohol, cetearyl alcohol alcohol, myristyl alcohol, behenyl alcohol, arachidyl alcohol, and mixtures thereof.

Nonlimiting examples of liquid fatty alcohols include isostearyl alcohol, oleyl alcohol, octyldodecanol, linoleyl alcohol, linolenyl alcohol, lauryl alcohol, 2-hexyldecanol, 2-heptyldecanol, enanthic alcohol (1-heptanol), caprylic alcohol (1-octanol), pelargonic alcohol (1-nonanol), capric alcohol (1-decanol), or mixtures thereof. In a preferred embodiment, the one or more liquid fatty alcohols are selected from lauryl alcohol, oleyl alcohol, octyldodecanol, and mixtures thereof.

Nonlimiting examples of low polar oils having a Polarity Index of about 23 mN/m to about 40 mN/m include fatty carbonates, fatty ethers, liquid linear or branched carboxylic acid esters, and mixtures thereof. More specific but nonlimiting examples include isopropyl palmitate, isopropyl myristate, coco-caprylate/caprate, isopropyl isostearate, butylene glycol dicaprylate/dicaprate, dicaprylyl carbonate, isopropyl stearate, ethylhexyl cocoate, dibutyl adipate, isodecyl neopentanoate, dicaprylyl ether, and mixtures thereof.

The hair treatment composition, in various embodiments, includes one or more water soluble solvents selected from glycerin, polyols, glycols, or mixtures thereof. More specific but nonlimiting examples include glycerin, ethylene glycol, propylene glycol, butylene glycol, caprylyl glycol, dipropylene glycol, and mixtures thereof.

The hair treatment composition, in various embodiments, includes one or more non-polymeric mono-, di-, and/or tri-carboxylic acids. The carboxylic acids are useful for adjusting the pH of the composition. Nonlimiting examples of non-polymeric, mono-, di-, and/or tri-carboxylic acids include lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, benzoic acid, citric acid, and mixtures thereof. In a preferred embodiment, the one or more carboxylic acid is lactic acid, tartaric acid, or mixtures thereof, especially in combination with fatty dimethylamines such as, for example, stearamidopropyl dimethylamine and/or brassicamidopropyl dimethylamine.

In a preferred embodiment, the hair treatment composition has a pH of from about 3 to about 8. Even more preferred is a pH of from about 5 to about 8.

In a preferred embodiment, the hair treatment composition is an oil-in-water emulsion.

The hair treatment composition may optionally include (or exclude) one or more fatty compounds other than (b), (c), and (d). Nonlimiting examples of such fatty compounds include nonpolar oils, polar oils, alkanes (paraffins), fatty alcohol derivatives, fatty acid derivatives, esters of fatty alcohols, hydroxy-substituted fatty acids, waxes, triglyceride compounds, and a mixture thereof.

In various embodiments, the hair treatment composition includes one or more cationic conditioning polymers. Nonlimiting examples include polyquaterniums (e.g., polyquaternium-4, polyquaternium-10, and polyquaternium-24), guar hydroxypropyltrimonium chloride, guar hydroxypropyltrimonium chloride, cocodimonium hydroxypropyl hydrolyzed rice protein, stearyldimoniumhydroxypropyl hydrolyzed rice protein, hydroxypropyltrimoniumhydrolyzed silk, cocodimonium hydroxypropyl hydrolyzed soy protein, lauryldimonium hydroxypropyl hydrolyzed soy protein, hydroxypropyltrimonium hydrolyzed soy protein, hydroxypropyltrimonium hydrolyzed vegetable protein, stearyldimonium hydroxypropyl hydrolyzed vegetable protein, cocodimonium hydroxypropyl hydrolyzed wheat protein, hydroxypropyltrimonium hydrolyzed wheat protein, stearyldimonium hydroxypropyl hydrolyzed wheat protein, and a mixture thereof.

The hair treatment compositions optionally include a variety of additional miscellaneous ingredients. Nonlimiting examples include preservatives, fragrances, pH adjusters, salts, chelating agents, buffers, antioxidants, flavonoids, vitamins, amino acids, emollients, botanical extracts, UV filtering agents, peptides, proteins, protein hydrolysates, protein isolates, fillers (e.g., organic and/or inorganic fillers such as talc, calcium carbonate, silica, particular materials, etc.), composition colorants, and mixtures thereof.

The hair treatment compositions of the present disclosure do not require nonionic emulsifiers for stability. Nonionic surfactants or emulsifiers may optionally be included in the hair treatment compositions, but they are not required to emulsify or stabilize the compositions. Thus, the hair treatment compositions, in some embodiments, may be free or essentially free from nonionic surfactants and emulsifiers.

The hair treatment compositions of the present disclosure do not require silicones, which are notorious for their detangling, slip, and smoothing properties. Silicones may optionally be included in the hair treatment compositions, but they are not required to provide detangling, slip, and smoothing properties. Thus, in various embodiments, the hair treatment compositions are free or essentially free from silicones. Despite the lack of silicones, the inventors discovered the claimed compositions perform surprisingly and significantly better with respect to detangling, ease of passing fingers, moisturization/hydration, smoothness, and shine when compared to detangling products containing silicones.

In various embodiments, the hair treatment compositions are free or essentially free from anionic surfactants, amphoteric surfactants, and/or C₂-C₆ monoalcohols, such as ethanol and isopropanol.

The present disclosure includes methods for treating hair. The hair treatment compositions can be used as “rinse off” (or “rinse out”) compositions or can be used as “leave on” (or “leave-in”) compositions. The methods for treating hair include applying a hair treatment composition of the present disclosure to the hair. The hair treatment composition may be applied to wet hair or dry hair. The hair treatment compositions are particularly useful in methods for detangling hair, especially curly hair.

The hair treatment composition is applied to the tangled hair, spread throughout the tangled hair, for example, using one's fingers, a comb, or a detangling utensil, like a detangling comb or detangling brush, and the hair is detangled. After detangling the hair, the hair treatment composition may optionally be rinsed from the hair and the hair optionally cleansed with a shampoo. In addition, or alternatively, the hair treatment compositions can be used as a post-cleansing (post-shampoo) detangling composition. After cleansing the hair, for example, with a cleansing composition like a shampoo, the cleansing composition/shampoo is typically rinsed from the hair. After rinsing, while the hair is still damp or wet, the hair treatment compositions of the present disclosure can be applied to the hair, and to the extent the hair is tangled, the hair treatment composition facilitates and expedites detangling of the hair. The hair treatment composition is preferably applied to the wet or damp hair shortly after cleansing the hair and that is detangled using one fingers, a comb, or a detangling utensil, like a like a detangling comb. After detangling the hair, the hair treatment composition can be optionally rinsed with water, or the hair treatment composition may optionally remain on the hair. In a preferred embodiment, the hair treatment composition remains on the hair (as a leave-in product). Upon drying, the hair has a natural look but enhanced shine, fiber alignment, and smoothness. In addition, the hair treatment composition used as a leave-on product does not form a whitish residue on the hair or flaking.

### BRIEF DESCRIPTION OF THE DRAWINGS

Implementation of the present technology is described, by way of example only, with reference to the attached figures, wherein:

FIG. 1 provides pictures of curl pattern 7 natural hair swatches treated with an inventive composition according to the present disclosure and curl pattern 7 natural hair swatches treated with comparative compositions; and

FIG. 2 provides a picture showing left-half and right-half of an individual's head of hair, wherein the left-half of the individual's head of hair was treated with an inventive composition according to the present disclosure and the right-half of the individual's head of hair was treated with a comparative composition.

The various aspects of the disclosure are not limited to the results, arrangements, and representations shown in the drawings.

### DETAILED DESCRIPTION OF THE DISCLOSURE

The instant disclosure relates to hair treatment compositions and to methods for treating hair using the hair-treatment compositions. Treatment with the compositions leads to improved manageability of hair, especially curly hair, which is more likely to suffer from tangling. The hair treatment compositions ease and expedite the detangling of hair, improve fiber alignment, reduce frizz, and increase shine and moisturization/hydration of the hair. The hair treatment compositions, for example, include:
(a) one or more cationic surfactants;
(b) one or more solid fatty alcohols;
   wherein (a) and (b) are in a combined amount of about 2 to about 15 wt. %;
(c) one or more liquid fatty alcohols;
(d) one or more low polar oils having a Polarity Index of about 23 mN/m to about 40 mN/m;
   wherein (c) and (d) are in a combined amount of about 2 to about 15 wt. %; and
(e) water;
   wherein the combined amount of (c) and (d) and the combined amount of (a) and (b) are in a weight ratio of about 0.5:1 to about 3:1 (((c)+(d)):((a)+(b))); and
   all percentages by weight are based on a total weight of the composition.

In another embodiment, the hair treatment compositions include:
(a) about 0.1 to about 5 wt. % of one or more cationic surfactants;
(b) about 1 to about 10 wt. % of one or more solid fatty alcohols;
(c) about 0.3 to about 6 wt. % of one or more liquid fatty alcohols;
(d) about 0.3 to about 6 wt. % of one or more low polar oils having a Polarity Index of about 23 mN/m to about 40 mN/m;
   wherein a weight ratio of a combination of (c) and (d) to a combination of (a) and (b) is from about 0.5:1 to about 3:1 (((c)+(d)):((a)+(b))); and
(e) about 60 to about 92 wt. % of water;
   wherein all percentages by weight are based on a total weight of the composition.

### (a) Cationic Surfactants

The term “cationic surfactant” as used in the present disclosure is a surfactant that may be positively charged when it is contained in the hair treatment compositions according to the present disclosure. The cationic surfactant may bear one or more positive permanent charges or may contain one or more functional groups that are cationizable in the compositions.

Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, and mixtures thereof.

The one or more cationic surfactants may be selected from quaternary ammonium compounds, fatty dialkylamines, or mixtures thereof.

Nonlimiting examples of quaternary ammonium compounds include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and combinations thereof.

Nonlimiting examples of fatty dialkylamines include oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, salts thereof, and combinations thereof.

In various embodiments, the one or more cationic surfactants are preferably selected from cetrimonium chloride, behentrimonium chloride, behentrimonium methosulfate, stearamidopropyl dimethylamine, brassicamidopropyl dimethylamine or a mixture thereof.

A more exhaustive but nonlimiting list of cationic surfactants is provided later, under the heading “Cationic Surfactants.”

The amount of the one or more cationic surfactants in the hair treatment composition will vary, but typically the hair treatment compositions include from about 0.1 to about 5 wt. % of the one or more cationic surfactants, based on the total weight of the hair treatment composition. In further embodiments, the hair treatment composition includes from about 0.1 to about 4 wt. %, about 0.1 to about 3 wt. %, about 0.3 to about 5 wt. %, about 0.3 to about 4 wt. %, about 0.3 to about 3 wt. %, about 0.5 to about 5 wt. %, about 0.5 to about 4 wt. %, or about 0.5 to about 3 wt. %, about 0.8 to about 5 wt. %, about 0.8 to about 4 wt. %, about 0.8 to about 3 wt. %, about 1 to about 5 wt. %, about 1 to about 4 wt. %, or about 1 to about 3 wt. % of the one or more cationic surfactants, based on the total weight of the hair treatment composition.

### (b) Solid Fatty Alcohols

The term “solid fatty alcohol” means a fatty alcohol having a melting point greater than 27° C. at atmospheric pressure (1.013×10⁵ Pa). For the purposes of the present disclosure, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name “MDSC 2920” by the company TA Instruments.

The term “fatty alcohol” means a long-chain aliphatic alcohol comprising from 8 to 40 carbon atoms, preferably from 12 to 34 or even from 12 to 30 carbon atoms and comprising at least one hydroxyl group OH. These fatty alcohols are neither oxyalkylenated nor glycerolated. Preferably, the solid fatty alcohols are of structure R—OH with R denoting a linear alkyl group, optionally substituted with one or more hydroxyl groups, comprising from 12 to 40, better still from 12 to 34, or even from 12 to 30, and most preferentially from 12 to 24 carbon atoms.

Nonlimiting examples of solid fatty alcohols include myristyl alcohol (1-tetradecanol), cetyl alcohol (1-hexadecanol), stearyl alcohol (1-octadecanol), arachidyl alcohol (1-eicosanol), behenyl alcohol (1-docosanol), lignoceryl alcohol (1-tetracosanol), ceryl alcohol (1-hexacosanol), montanyl alcohol (1-octacosanol), myricyl alcohol (1-triacontanol), and mixtures thereof. In a preferred embodiment, the one or more solid fatty alcohols are selected from myristyl alcohol, cetyl alcohol, stearyl alcohol, behenyl alcohol and mixtures thereof, such as cetylstearyl alcohol, cetearyl alcohol, or mixtures thereof.

The total amount of the one or more solid fatty alcohols in the hair treatment compositions will vary, but is typically from about 1 to about 10 wt. %, based on the total weight of the hair treatment composition. In further embodiments, the hair treatment composition includes about 1 to about 9 wt. %, about 1 to about 8 wt. %, about 1 to about 7 wt. %, about 2 to about 10 wt. %, about 2 to about 9 wt. %, about 2 to about 8 wt. %, about 3 to about 10 wt. %, about 3 to about 9 wt. %, about 3 to about 8 wt. %, about 4 to about 10 wt. %, about 4 to about 9 wt. %, about 4 to about 8 wt. %, about 5 to about 10 wt. %, about 5 to about 9 wt. %, or about 5 to about 8 wt. % of the one or more solid fatty alcohols, based on the total weight of the hair treatment composition.

### Combined Amounts of (a) and (b)

The combined amount of the one or more cationic surfactants of (a) and the one or more solid fatty alcohols of (b) in the hair treatment composition will vary, but is typically from about 2 to about 15 wt. %, based on the total weight of the hair treatment composition. In further embodiments, the combined amount of (a) and (b) in the hair treatment composition is from about 2 to about 12 wt. %, about 2 to about 10 wt. %, about 2 to about 8 wt. %, about 3 to about 15 wt. %, about 3 to about 12 wt. %, about 3 to about 10 wt. %, about 3 to about 12 wt. %, about 4 to about 15 wt. %, about 4 to about 12 wt. %, about 4 to about 10 wt. %, about 4 to about 8 wt. %, about 5 to about 15 wt. %, about 5 to about 12 wt. %, about 5 to about 10 wt. %, or about 5 to about 8 wt. %, based on the total weight of the hair treatment composition.

### (c) Liquid Fatty Alcohols

The term “liquid fatty alcohol” means a fatty alcohol having a melting point of less than or equal to 27° C. at atmospheric pressure (1.013×10⁵ Pa). For the purposes of the present disclosure, the melting point corresponds to the temperature of the most endothermic peak observed on thermal analysis (differential scanning calorimetry or DSC) as described in the standard ISO 11357-3; 1999. The melting point may be measured using a differential scanning calorimeter (DSC), for example the calorimeter sold under the name “MDSC 2920” by the company TA Instruments.

The liquid fatty alcohols may be linear or branched, saturated or unsaturated alcohols, preferably unsaturated or branched alcohols, including from 8 to 40 carbon atoms and preferably from 8 to 30 carbon atoms. These fatty alcohols are neither oxyalkylenated nor glycerolated. Nonlimiting examples include octyldodecanol, 2-butyloctanol, 2-hexyldecanol, 2-undecylpentadecanol, isostearyl alcohol, oleyl alcohol, linolenyl alcohol, ricinoleyl alcohol, undecylenyl alcohol, linoleyl alcohol, isostearyl alcohol, octyldodecanol, lauryl alcohol, 2-hexyldecanol, 2-heptyldecanol, caproic alcohol (1-hexanol), enanthic alcohol (1-heptanol), caprylic alcohol (1-octanol), pelargonic alcohol (1-nonanol), capric alcohol (1-decanol), or mixtures thereof. In a preferred embodiment, the one or more liquid fatty alcohols are selected from octyldodecanol, isostearyl alcohol, lauryl alcohol, oleyl alcohol, linolenyl alcohol, ricinoleyl alcohol, undecylenyl alcohol, linoleyl alcohol, and mixtures thereof.

The total amount of the one or more liquid fatty alcohols in the hair treatment composition will vary, but is typically from about 0.1 to about 6 wt. %, based on the total weight of the hair treatment composition. In further embodiments, the hair treatment composition includes about 0.1 to about 5 wt. %, about 0.1 to about 4 wt. %, about 0.1 to about 3 wt. %, about 0.2 to about 6 wt. %, about 0.2 to about 5 wt. %, about 0.2 to about 4 wt. %, about 0.2 to about 3 wt. %, about 0.5 to about 6 wt. %, about 0.5 to about 5 wt. %, about 0.5 to about 4 wt. %, or about 0.5 to about 3 wt. % of the one or more liquid fatty alcohols, based on the total weight of the hair treatment composition.

### (d) Low Polar Oils

A “low polar oil” is an oil having a Polarity Index (a measure of interfacial tension) of about 23 mN/m to about 40 mN/m. A “low polar oil” may alternatively be referred to as a “medium polar oil.” The polarity of an oil is defined as the polarity index (interfacial tension) of the oil with respect to water. The polarity may be determined using a ring tensiometer (e.g., Krüss K 10), which measures the interfacial tension in mN/m in analogy to the ASTM method D971-99a (2004).

Interfacial tension is the force which acts on an imaginary line one meter in length in the interface between two phases. The physical unit for this interfacial tension is conventionally calculated from the force/length relationship and is usually expressed in mN/m (millinewtons divided by meters). For the purposes of the present disclosure, nonpolar oils have an interfacial tension (Polarity Index) of greater than 40 mN/m, low polar oils (or mid-polar oils) have an interfacial tension (Polarity Index) of 23 mN/m to 40 mN/m, and polar oils have an interfacial tension (Polarity Index) less than 23 mN/m. Nonlimiting examples of nonpolar oils, low polar oils (or mid-polar oils), and polar oils is provided below, in Table A.

Table A includes the Polarity Index for various oils, as a point of reference.

TABLE A

| | |
|---|---|
| | Polarity Index |
| Oil (CTFA Name) | [mN/m] |

Nonpolar Oils (>40 mN/m)

| | |
|---|---|
| Isoparaffin (C12-C14) | 53.0 |
| Cycloparaffin | 49.1 |
| Polydecene | 46.7 |
| Polydimethylsiloxane | 46.5-40.9 ¹ |
| Squalane (Squalan ®) | 46.2 |
| Hydrogenated polyisobutene (Luvitol | 44.7 |
| Lite ®) | |
| Isohexadecane (ARLAMOL ® ND) | 43.8 |
| Mineral oil (Paraffin oil perliquidum) | 43.7 |
| Isoeicosane | 41.9 |
| Ethoxydiglycol Oleate | 40.5 |
| Decyl Olivate | 40.3 |

Low Polar Oils (23 mN/m-40 mN/m)

| | |
|---|---|
| Dioctylcyclohexane | 39.0 |
| Mineral Oil (Pionier ® 2071) | 38.3 |
| Isocetyl Palmitate (Tegosoft ® HP) | 36.2 |
| Isocetyl Palmitate (Isofol Ester ® 1693) | 33.5 |
| Cyclopentasiloxane | 32.3 |
| Dicaprylyl carbonate | 31.7 |
| Octyl Isostearate | 31.6 |
| Trimethylhexyl Isononanoate | 31.1 |
| 2-Ethylhexyl Isononanoate | 31.0 |
| Dicaprylyl ether | 30.9 |
| Dihexyl carbonate | 30.9 |
| Polydecene | 30.1 |
| Octyl Cocoate | 30.0 |
| Isodecyl Neopentanoate | 29.9 |
| Isohexy Decanoate (Dermol ® IHD) | 29.7 |
| Isodecyl Octanoate ( Dermol ® 108) | 29.6 |
| Dihexyl Ether | 29.2 |
| Isodecyl 3,5,5 Trimethyl Hexanoate | 29.1 |
| (Dermol 109) | |
| Oleyl Erucate (Cetiol J600 ®) | 27.1 |
| Passiflora Incarnata Oil (Cegesoft ® PFO) | 27.2 |
| Jojoba oil | 26.2 |
| Octyl palmitate | 23.1 |
| Macadamia nut oil | 22.1 |
| Isopropyl stearate | 21.9 |
| Rapeseed oil | 21.9 |
| Hexyl Decanol (Isofol ® 16) | 24.3 |
| Isotridecyl 3,5,5 Trimethylhexanonanoate | 24.5 |
| (Dermol ® 139) | |
| Mixed decanoyl and octanoyl glycerides | 25.3 |
| (Myritol ® 312) | |
| 2-Ethylhexanoic Acid 3,5,5 Trimethyl | 26.2 |
| Ester (Dermol ® 98) | |
| Cetystearyl octanoate | 28.6 |
| Dimethicone (silicon oil 20 ct) | 26.6 |
| Isopropyl palmitate | 25.2 |
| Octyldodecanol | 24.8 |
| Dioctyl adipate (ARLAMOL ® DOA) | 24.5 |
| Isopropyl myristate | 24.2 |
| Octyl palmitate (2-ethylhexyl palmitate) | 23.1 |

Polar Oils (<23 mN/m)

| | |
|---|---|
| Octyldodeceyl Myristate | 22.1 |
| Butyl Octanoic Acid (ISOCARB ® 12) | 22.1 |
| Isopropyl stearate | 21.9 |
| Caprylic/capric triglycerides | 21.3 |
| Isopropyl isostearate | 21.2 |
| Jojoba oil | 20.8 |
| Cyclomethicone (ARLAMOL ® D4) | 20.6 |
| Groundnut oil | 20.5 |
| Almond oil | 20.3 |
| Sunflower oil | 19.3 |
| Decyl oleate | 18.7 |
| Avocado oil | 18.3 |
| Olive oil | 16.9 |
| Dibutyl adipate | 14.3 |
| Castor oil | 13.7 |
| Calendula oil | 11.1 |
| Wheatgerm oil | 8.3 |
| Almond oil | 20.3 |
| Sunflower oil | 19.3 |
| Decyl oleate | 18.7 |
| Avocado oil | 18.3 |
| Olive oil | 16.9 |
| Castor oil | 13.7 |
| Calendula oil | 11.1 |
| Wheatgerm oil | 8.3 |
| Propylene glycol monoisostearate | 6.2 |
| Cocoglycerides | 5.1 |

¹ Polydimethylsiloxane polymers have different polarity depending on polymer length.

In various embodiments, the “low polar oil” is preferably a non-silicone oil. The term “non-silicone oil” means an oil that does not contain any silicone (Si) atoms. Thus, the term “non-silicone, low polar oil” means an oil that does not contain any silicone (Si) atoms and has a Polarity Index of about 23 mN/m to about 40 mN/m.

Nonlimiting examples of non-silicone, low polar oils include dioctylcyclohexane, isocetyl palmitate, isocetyl palmitate, dicaprylyl carbonate, octyl Isostearate, trimethylhexyl isononanoate, 2-ethylhexyl Isononanoate, dicaprylyl ether, dihexyl carbonate, polydecene, octyl cocoate, isodecyl neopentanoate, isohexy decanoate, isodecyl octanoate, dihexyl ether, isodecyl 3,5,5 trimethyl hexanoate, oleyl erucate, Passiflora incarnata oil, Jojoba oil, octyl palmitate, macadamia nut oil, isopropyl stearate, rapeseed oil, hexyl decanol, isotridecyl 3,5,5 trimethylhexanonanoate, 2-ethylhexanoic acid 3,5,5 trimethyl ester, cetystearyl octanoate, isopropyl palmitate, octyldodecanol, dioctyl adipate, isopropyl myristate, octyl palmitate (2-ethylhexyl palmitate), isopropyl isostearate, coco-caprylate/caprate, butylene glycol dicaprylate/dicaprate, ethylhexyl cocoate, dibutyl adipate, isodecyl neopentanoate, and mixtures thereof.

More generally, the one or more low polar oils may be selected from fatty esters, fatty ethers, propylene glycol fatty acid esters, fatty carbonate esters, and mixtures thereof. In preferred embodiment, at least one of the one or more low polar oils is selected from fatty esters, fatty carbonate esters, or mixtures thereof.

Nonlimiting examples of fatty esters include isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate, 2-octyldodecyl lactate, di(2-ethylhexyl) succinate, diisostearyl malate, glycerine triisostearate, and diglycerine triisostearate, provided the fatty esters have a Polarity Index of about 23 mN/m to about 40 mN/m.

Nonlimiting examples of fatty ethers include stearyl ether, dicaprylyl ether, dicetyl ether distearyl ether, dodecyl ether, dilauryl ether, dimyristyl ether, diisononyl ether, or a combination thereof, provided the fatty ethers has a Polarity Index of about 23 mN/m to about 40 mN/m. In a preferred embodiment, the hair treatment composition includes dicaprylyl ether.

Nonlimiting examples of fatty carbonate esters include dialkyl carbonates of formula: R₁O(C═O)OR₂, wherein R₁ and R₂ are independently linear or branched, saturated or unsaturated alkyl chains having 1 to 30 carbon atoms, or having 2 to 28 carbon atoms, or having 4 to 25 carbon atoms, or having 6 to 22 carbon atoms, preferably one or more fatty carbonates selected from C14-15 dialkyl carbonate, dicaprylyl carbonate, diethyl carbonate, dihexyl carbonate, diethylhexyl carbonate, dimethoxyphenyl phenyloxoethyl ethylcarbonate, dimethyl carbonate, dipropyl carbonate, dipropylheptyl carbonate, dioctyl carbonate, and a combination thereof, provided the fatty carbonate esters have a Polarity Index of about 23 mN/m to about 40 mN/m. In a preferred embodiment, the hair treatment composition includes dicaprylyl carbonate.

In a preferred embodiment, the hair treatment composition includes one or more low polar oils selected from dicaprylyl carbonate, dicaprylyl ether, or a mixture thereof.

The total amount of the one or more low polar oils in the hair treatment composition will vary but is typically from about 0.3 to about 8 wt. %, based on the total weight of the hair treatment composition. In further embodiments, the hair treatment composition includes about 0.3 to about 7 wt. %, about 0.3 to about 6 wt. %, about 0.3 to about 5 wt. %, about 0.3 to about 4 wt. %, about 0.5 to about 8 wt. %, about 0.5 to about 7 wt. %, about 0.5 to about 6 wt. %, about 0.5 to about 5 wt. %, about 0.5 to about 4 wt. %, about 1 to about 8 wt. %, about 1 to about 7 wt. %, about 1 to about 6 wt. %, about 1 to about 5 wt. %, about 1 to about 4 wt. %, about 2 to about 8 wt. %, about 2 to about 6 wt. %, about 2 to about 5 wt. %, about 2 to about 4 wt. %, about 2.5 to about 8 wt. %, about 2.5 to about 6 wt. %, about 2.5 to about 5 wt. %, or about 2.5 to about 4 wt. % of the one or more low polar oils, based on the total weight of the hair treatment composition.

### Combined Amounts of (c) and (d)

The combined amount of the one or more liquid fatty alcohols of (c) and the one or more low polar oils having a Polarity Index of about 23 mN/m to about 40 mN/m of (d) in the hair treatment composition will vary, but is typically from about 2 to about 15 wt. %, based on the total weight of the hair treatment composition. In further embodiments, the combined amount of (c) and (d) in the hair treatment composition is from about 2 to about 12 wt. %, about 2 to about 10 wt. %, about 2 to about 8 wt. %, about 20 to about 7 wt. %, about 2 to about 6 wt. %, about 3 to about 12 wt. %, about 3 to about 10 wt. %, about 3 to about 8 wt. %, about 3 to about 7 wt. %, about 3 to about 6 wt. %, about 4 to about 15 wt. %, about 4 to about 12 wt. %, about 4 to about 10 wt. %, about 4 to about 8 wt. %, about 4 to about 7 wt. %, or about 4 to about 6 wt. %, based on the total weight of the hair treatment composition.

### Weight Ratio (Combination of (c) and (d) to Combination of (a) and (b))

The weight ratio of the combination of (c) and (d) to the combination of (a) and (b) will vary but is typically from about 0.5:1 to about 3:1 (((c)+(d)):((a)+(b))). In further embodiments, the weight ratio of ((c)+(d)) to ((a)+(b)) is from about 0.5:1 to about 2:1, about 0.5:1 to about 1.5:1, about 0.5:1 to about 1:1, about 0.5:1 to about 0.9:1, about 0.6:1 to about 3:1, about 0.6:1 to about 2:1, about 0.6:1 to about 1.5:1, about 0.6:1 to about 1:1, or about 0.6:1 to about 0.9:1 (((c)+(d)):((a)+(b))).

### (e) Water

The hair treatment compositions of the present disclosure include a sizeable amount of water. The total amount of water in the hair treatment composition will vary but is typically at least 50 wt. %, based on the total weight of the hair treatment compositions. In various embodiments, the hair treatment composition includes from about 55 to about 92 wt. % of water, based on the total weight of the hair treatment composition. In further embodiments, the hair treatment composition includes from about 60 to about 92 wt. %, about 65 to about 92 wt. %, about 70 to about 92 wt. %, about 75 to about 92 wt. %, about 80 to about 92 wt. %, about 55 to about 90 wt. %, about 60 to about 90 wt. %, about 65 to about 90 wt. %, about 70 to about 90 wt. %, about 75 to about 90 wt. %, or about 80 to about 90 wt. %, based on the total weight of the hair treatment composition.

### (f) Water Soluble Solvents

The term “water soluble solvent” is interchangeable with the terms “water soluble organic solvent” and “water-miscible solvent” and means a compound that is liquid at 25° C. and at atmospheric pressure (760 mmHg), and it has a solubility of at least 50% in water under these conditions. In some cases, the water-soluble solvents have a solubility of at least 60%, 70%, 80%, or 90%. Non-limiting examples of water-soluble solvents include, for example, C₂-C₆ monoalcohols, glycerin, polyols, glycols, glycol ethers, and mixtures thereof.

Nonlimiting examples of C₂-C₆ monoalcohols and polyols include ethanol, isopropyl alcohol, propyl alcohol, 1-butanol, 2-butanol, 2-methylpropan-1-ol, 2-methylpropanol, and mixtures thereof.

Water soluble solvents include glycols and glycol ethers such as, for example, monomethyl, monoethyl and monobutyl ethers of ethylene glycol, propylene glycol, such as, for example, monomethyl ether of propylene glycol, butylene glycol, hexylene glycol, dipropylene glycol as well as alkyl ethers of diethylene glycol, for example monoethyl ether or monobutyl ether of diethylene glycol.

Water soluble solvents include polyols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, and a mixture thereof.

Water soluble solvents are not necessarily required in the hair treatment compositions of the present disclosure and therefore, is some cases, the hair treatment composition may be free or essentially free from water soluble solvents. Nonetheless, in certain embodiments, use of one or more water soluble solvents is preferred. For example, in a preferred embodiment, the hair treatment compositions include one or more polyols selected from glycerin, propylene glycol, butylene glycol, pentylene glycol, hexylene glycol, caprylyl glycol, dipropylene glycol, and mixtures thereof.

In various embodiments, the hair treatment composition is preferably free or essentially free from ethanol. In further preferred embodiments, the hair treatment composition is free or essentially free from ethanol and isopropanol. In an even more preferred embodiment, the hair treatment composition is free or essentially free from C₂-C₆ monoalcohols.

The total amount of the one or more water soluble solvents in the hair treatment compositions, if present, can vary but is typically from about 0.01 to about 15 wt. %, based on the total weight of the hair treatment composition. In further embodiments, the hair treatment compositions include about 0.01 to about 10 wt. %, about 0.01 to about 8 wt. %, about 0.01 to about 5 wt. %, about 0.05 to about 15 wt. %, about 0.05 to about 10 wt. %, about 0.05 to about 8 wt. %, about 0.05 to about 5 wt. %, about 0.1 to about 15 wt. %, about 0.1 to about 10 wt. %, about 0.1 to about 8 wt. %, about 0.1 to about 5 wt. % of the one or more water soluble solvents, based on the total weight of the hair treatment composition.

(g) Mono-, Di-, and/or Tri-Carboxylic Acids

For purposes of the present disclosure, the mono-, di-, and/or tri-carboxylic acids are non-thiol, non-polymeric, mono-, di-, and/or tri-carboxylic acids. Nonlimiting examples include formic acid, acetic acid, lactic acid, propionic acid, butyric acid, gluconic acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, and arachidic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, and 2,6-naphthalene dicarboxylic acid, citric acid, isocitric acid, aconitric acid, propane-1,2,3-tricarboxylic acid, and benzene-1,3,5-tricarboxylic acid, a salt thereof, and mixtures thereof.

Nonlimiting examples of monocarboxylic acids include formic acid, acetic acid, lactic acid, propionic acid, butyric acid, gluconic acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, and arachidic acid, salts thereof, and mixtures thereof.

Nonlimiting examples of dicarboxylic acids include oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, and 2,6-naphthalene dicarboxylic acid, salts thereof, and mixtures thereof.

Nonlimiting examples of tricarboxylic acids include citric acid, isocitric acid, aconitric acid, propane-1,2,3-tricarboxylic acid, and benzene-1,3,5-tricarboxylic acid, salts thereof, and mixtures thereof.

In a preferred embodiment, the mono-, di-, and/or tri-carboxylic acids are selected from mono-, di-, and/or tri-carboxylic acids having from 1 to 9 carbon atoms.

The total amount of the mono-, di-, and/or tri-carboxylic acids in the hair treatment composition will vary, for example, based on the desired pH of the hair treatment composition. Nonetheless, the hair treatment composition typically includes about 0.01 to about 5 wt. % of the mono-, di-, and/or tri-carboxylic acids, based on the total weight of the hair treatment composition. In further embodiments, the hair treatment composition includes about 0.01 to about 3 wt. %, about 0.01 to about 2 wt. %, about 0.05 to about 5 wt. %, about 0.05 to about 3 wt. %, about 0.05 to about 2 wt. %, about 0.1 to about 5 wt. %, about 0.1 to about 3 wt. %, or about 0.1 to about 2 wt. % of the one or more mono-, di-, and/or tri-carboxylic acids, based on the total weight of the hair treatment composition.

(h) Fatty Compounds Other than (b), (c), and (d)

The hair treatment compositions may optionally include one or more fatty compounds other than the solid fatty alcohols of (b), the liquid fatty alcohols of (c), and the low polar oils of (d). The term “fatty compounds” is interchangeable with the term, “fatty material.” Fatty compounds are known as compounds that are not soluble (or only sparingly soluble) in water; they are hydrophobic and are often solubilized in organic solvents. A fatty compound is “sparingly soluble” when not more than 0.5 grams of the fatty compound can be dissolved in 1 liter of water at 25° C.

Fatty compounds other than (b), (c), and (d) include materials such as oils, fats, waxes, hydrocarbons, fatty esters, fatty acids, silicones, etc. Non-limiting examples of useful fatty compounds include oils, waxes, alkanes (paraffins), fatty acids, fatty esters, triglyceride compounds, lanolin, hydrocarbons, derivatives thereof, silicones, and mixtures thereof, provided they are not the fatty alcohols of (b) and (c) and not the low polar oils of (d). Fatty compounds are described by the International Federation Societies of Cosmetic Chemists, for example, in Cosmetic Raw Material Analysis and Quality, Volume I: Hydrocarbons, Glycerides, Waxes and Other Esters (Redwood Books, 1994), which is incorporated herein by reference in its entirety.

### Fatty Alcohol Derivatives

The fatty compounds may include one or more fatty alcohol derivatives, which are different from fatty alcohols (I.e., different from solid fatty alcohols of (b) and liquid fatty alcohols of (c)). Fatty alcohol derivatives include fatty esters derived from one or more fatty alcohols. Fatty alcohol derivatives also include alkoxylated fatty alcohols, e.g., having about 1 to about 100 moles of an alkylene oxide per mole of alkoxylated fatty alcohol. For example, the alkoxylated fatty alcohols may be alkoxylated with about 1 to about 80 moles, about 2 to about 50, about 5 to about 45 moles, about 10 to about 40 moles, or 15 to about 35 mores, including all ranges and subranges therebetween, of an alkylene oxide per mole of alkoxylated fatty alcohol.

As examples of alkoxylated fatty alcohols, steareth (for example, steareth-2, steareth-20, and steareth-21), laureth (for example, laureth-4, and laureth-12), ceteth (for example, ceteth-10 and ceteth-20) and ceteareth (for example, ceteareth-2, ceteareth-10, and ceteareth-20) are mentioned. In at least one instance, the one or more alkoxylated fatty alcohols include steareth-20. In some instances, the one or more alkoxylated fatty alcohols may be exclusively steareth-20.

Additional fatty alcohol derivatives that may, optionally be suitable include methyl stearyl ether; 2-ethylhexyl dodecyl ether; stearyl acetate; cetyl propionate; the ceteth series of compounds, such as ceteth-1 through ceteth-45, which are ethylene glycol ethers of cetyl alcohol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof.

### Fatty Acids and Fatty Acid Derivatives

In some instances, the fatty compounds may be chosen from fatty acids, fatty acid derivatives, esters of fatty acids, hydroxyl-substituted fatty acids, and alkoxylated fatty acids. The fatty acids may be straight or branched chain acids and/or may be saturated or unsaturated. Non-limiting examples of fatty acids include diacids, triacids, and other multiple acids as well as salts of these fatty acids. For example, the fatty acid may optionally include or be chosen from lauric acid, palmitic acid, stearic acid, behenic acid, arichidonic acid, oleic acid, isostearic acid, sebacic acid, and a mixture thereof. In some cases, the fatty acids are selected from the group consisting of palmitic acid, stearic acid, and a mixture thereof.

### Fatty Acid Derivatives Include Polyglycerol Esters of Fatty Acids.

The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substituted fatty acids, and a mixture thereof. Non-limiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof. Preferred for use herein are glycerol monostearate, 12-hydroxy stearic acid, and a mixture thereof.

### Waxes

The fatty compounds may, in some instances, include one or more waxes. Non-limiting examples of waxes in this category include for example, synthetic wax, ceresin, paraffin, ozokerite, polyethylene waxes, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, acacia decurrents flower wax, vegetable waxes (such as sunflower seed (Helianthus annuus), carnauba, candelilla, ouricury or japan wax or cork fibre or sugarcane waxes), or a mixture thereof.

### Oils

In some instances, the fatty compounds may include or be chosen from one or more oil(s), provided the oil is not a low polarity oil. Suitable oils include, but are not limited to, natural oils, hydrocarbons, esters, diesters, and triesters.

### Silicones

Non-limiting examples of silicones include amine-functionalized silicones (e.g., amodimethicone), dimethicone, bis-aminopropyl dimethicone, trimethyl silylamodimethicone, dimethicone copolyols, etc. The hair cosmetic composition may include, in some instances, one or more silicones chosen from polydimethylsiloxanes (dimethicones), polydiethylsiloxanes, polydimethyl siloxanes having terminal hydroxyl groups (dimethiconols), polymethylphenylsiloxanes, phenylmethylsiloxanes, amino functional polydimethylsiloxane (amodimethicone), bis-aminopropyl dimethicone, trimethylsilylamodimethicone, dimethicone copolyols, dimethicone copolyol esters, dimethicone copolyol quaternium nitrogen containing compounds, dimethicone copolyol phosphate esters, and mixtures thereof. For example, the one or more silicones may be or include one or more dimethicone copolyols. The copolyols may be chosen from dimethicone PEG-8 Adipate, dimethicone PEG-8 benzoate, dimethicone PEG-7 Phosphate, dimethicone PEG-10 phosphate, dimethicone PEG/PPG-20/23 benzoate, dimethicone PEG/PPG-7/4 Phosphate, dimethicone PEG/PPG-12/4 phosphate, PEG-3 dimethicone, PEG-7 dimethicone, PEG-8 dimethicone, PEG-9 dimethicone, PEG-10 dimethicone, PEG-12 dimethicone, PEG-14 dimethicone, PEG-17 dimethicone, PEG/PPG-3/10 dimethicone, PEG/PPG-4/12 dimethicone, PEG/PPG-6/11 dimethicone, PEG/PPG-8/14 dimethicone, PEG/PPG-14/4 dimethicone, PEG/PPG-15/15 Dimethicone, PEG/PPG-16/2 dimethicone, PEG/PPG-17/18 dimethicone, PEG/PPG-18/18 dimethicone, PEG/PPG-19/19 dimethicone, PEG/PPG-20/6 dimethicone, PEG/PPG-20/15 dimethicone, PEG/PPG-20/20 dimethicone, PEG/PPG-20/23 dimethicone, PEG/PPG-20/29 dimethicone, PEG/PPG-22/23 dimethicone, PEG/PPG-22/24 dimethicone, PEG/PPG-23/6 dimethicone, PEG/PPG-25/25 dimethicone, PEG/PPG-27/27 dimethicone, and mixtures thereof.

The silicones may be functionalized with an amino group or functionalized with a methacrylic group. The term “amino-functionalized silicone” or “amino silicones” means a silicone containing at least one primary amino, secondary amino, tertiary amino and/or quaternary ammonium group. The structure of the amino-functionalized silicone may be linear or branched, cyclic or non-cyclic. The amino functional group may be at any position in the silicone molecule, preferably at the end of the backbone (for example, in the case of amodimethicone) and/or in the side chain.

The silicone compounds may be selected from those having at least one quaternary ammonium group. Suitable non-limiting examples are quaternium 80, silicone quaternium-1, silicone quaternium-2, silicone quaternium-2 panthenol succinate, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-16, silicone quaternium-16/Glycidoxy Dimethicone Crosspolymer, silicone quaternium-17, silicone quaternium-18, silicone quaternium-20 and silicone quaternium-21. Preferred are quaternium 80, silicone quaternium-16, silicone quaternium-18, silicone quaternium-1, silicone quaternium-2, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-17, silicone quaternium-20 and silicone quaternium-21. More preferred are quaternium 80, silicone quaternium-16, silicone quaternium-18, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, and silicone quaternium-17.

Although the hair treatment composition may optionally include one or more silicones, as outlined above, in a preferred embodiment, the hair treatment compositions are free or essentially free from silicones, such as those outlined above.

The total amount of fatty compounds in the hair treatment compositions, if present, may vary. For example, the total amount of fatty compounds may be about 0.01 to about 20 wt. %, about 0.01 to about 15 wt. %, about 0.01 to about 10 wt. %, about 0.01 to about 5 wt. %, about 0.01 to about 3 wt. %, about 0.1 to about 20 wt. %, about 0.1 to about 15 wt. %, about 0.1 to about 10 wt. %, about 0.1 to about 5 wt. %, about 0.1 to about 3 wt. %, about 1 to about 20 wt. %, about 1 to about 15 wt. %, about 1 to about 10 wt. %, about 1 to about 5 wt. %, or about 1 to about 3 wt. %, including any combination, sub-combination, range, or sub-range thereof by weight, based on the total weight of the hair treatment composition.

### (i) Cationic Conditioning Polymers

Cationic polymers for purposes of the instant disclosure are polymers bearing a positive charge or incorporating cationic entities in their structure. The cationic polymers can comprise mixtures of monomer units derived from amine- and/or quaternary ammonium-substituted monomer and/or compatible spacer monomers. Cationic polymers often provide conditioning benefits to the hair treatment compositions and therefore may be referred to as “cationic conditioning polymers.” Non-limiting examples of cationic polymers include copolymers of 1-vinyl-2-pyrrolidine and 1-vinyl-3-methyl-imidazolium salt (e.g., chloride salt) (referred to as Polyquaternium-16); copolymers of 1-vinyl-2-pyrrolidine and dimethylaminoethyl methacrylate (referred to as Polyquaternium-11); cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7); polysaccharide polymers, such as cationic cellulose derivatives and cationic starch derivatives. Cationic cellulose is available as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). Additionally or alternatively, the cationic conditioning polymers may include or be chosen from cationic guar gum derivatives, such as guar hydroxypropyltrimonium chloride.

Preferred cationic polymers include cationic polysaccharide polymers, such as cationic cellulose, cationic starch, and cationic guar gum. In the context of the instant disclosure, cationic polysaccharide polymers include cationic polysaccharides and polysaccharide derivatives (e.g., derivatized to be cationic), for example, resulting in cationic cellulose (cellulose derivatized to be cationic), cationic starch (derivatized to be cationic), or cationic guar (guar derivatized to be cationic).

Non-limiting examples of cationic celluloses include hydroxyethylcellulose (also known as HEC), hydroxymethylcellulose, methylhydroxyethylcellulose, hydroxypropylcellulose (also known as HPC), hydroxybutylcellulose, hydroxyethylmethylcellulose (also known as methyl hydroxyethylcellulose) and hydroxypropylmethylcellulose (also known as HPMC), cetyl hydroxyethylcellulose, polyquaternium-10, polyquaternium-24, and mixtures thereof, preferably polyquaternium-10, polyquaternium-24, and mixtures thereof.

Non-limiting examples of cationic guar include guar hydroxypropyltrimonium chloride, hydroxypropyl guar hydroxypropyltrimonium chloride, guar hydroxypropyltrimethylammonium chloride, and mixtures thereof.

Non-limiting examples of cationic starch include starch hydroxypropyltrimonium chloride, hydroxypropyl oxidized starch PG trimonium chloride, and a mixture thereof.

In certain embodiments, the hair treatment composition may include one or more polyquaterniums. Nonlimiting examples include polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof.

In certain embodiments, the hair treatment composition may include polyquaternium-1 (ethanol, 2,2′,2″-nitrilotris-, polymer with 1,4-dichloro-2-butene and N,N,N′,N′-tetramethyl-2-butene-1,4-diamine), polyquaternium-2, (poly[bis(2-chloroethyl) ether-alt-1,3-bis[3-(dimethylamino) propyl]urea]), polyquaternium-4, (hydroxyethyl cellulose dimethyl diallylammonium chloride copolymer; Diallyldimethylammonium chloride-hydroxyethyl cellulose copolymer), polyquaternium-5 (copolymer of acrylamide and quaternized dimethylammoniumethyl methacrylate), polyquaternium-6 (poly(diallyldimethylammonium chloride)), polyquaternium-7 (copolymer of acrylamide and diallyldimethylammonium chloride), polyquaternium-8 (copolymer of methyl and stearyl dimethylaminoethyl ester of methacrylic acid, quaternized with dimethylsulphate), polyquaternium-9 (homopolymer of N,N-(dimethylamino)ethyl ester of methacrylic acid, quaternized with bromomethane), polyquaternium-10 (quaternized hydroxyethyl cellulose), polyquaternium-11 (copolymer of vinylpyrrolidone and quaternized dimethylaminoethyl methacrylate), polyquaternium-12 (ethyl methacrylate/abietyl methacrylate/diethylaminoethyl methacrylate copolymer quaternized with dimethyl sulfate), polyquaternium-13 (ethyl methacrylate/oleyl methacrylate/diethylaminoethyl methacrylate copolymer quaternized with dimethyl sulfate), polyquaternium-14 (trimethylaminoethylmethacrylate homopolymer), polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), polyquaternium-19 (copolymer of polyvinyl alcohol and 2,3-epoxypropylamine), polyquaternium-20 (copolymer of polyvinyl octadecyl ether and 2,3-epoxypropylamine), polyquaternium-22 (copolymer of acrylic acid and diallyldimethylammonium chloride), polyquaternium-24 (auaternary ammonium salt of hydroxyethyl cellulose reacted with a lauryl dimethyl ammonium substituted epoxide), polyquaternium-27 (block copolymer of Polyquaternium-2 and Polyquaternium-17), polyquaternium-28 (copolymer of vinylpyrrolidone and methacrylamidopropyl trimethylammonium), polyquaternium-29 (chitosan modified with propylen oxide and quaternized with epichlorhydrin), polyquaternium-30 (ethanaminium, N-(carboxymethyl)-N, N-dimethyl-2-[(2-methyl-1-oxo-2-propen-1-yl)oxy]-, inner salt, polymer with methyl 2-methyl-2-propenoate), polyquaternium-31 (N,N-dimethylaminopropyl-N-acrylamidine quaternized with diethylsulfate bound to a block of polyacrylonitrile), polyquaternium-32 (poly(acrylamide 2-methacryloxyethyltrimethyl ammonium chloride)), polyquaternium-33 (copolymer of trimethylaminoethylacrylate salt and acrylamide), polyquaternium-34 (copolymer of 1,3-dibromopropane and N, N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), polyquaternium-36 (copolymer of N,N-dimethylaminoethylmethacrylate and buthylmethacrylate, quaternized with dimethylsulphate), polyquaternium-37 (poly(2-methacryloxyethyltrimethylammonium chloride)), polyquaternium-39 (terpolymer of acrylic acid, acrylamide and diallyldimethylammonium Chloride), polyquaternium-42 (poly[oxyethylene(dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), polyquaternium-44 (3-Methyl-1-vinylimidazolium methyl sulfate-N-vinylpyrrolidone copolymer), polyquaternium-45 (copolymer of (N-methyl-N-ethoxyglycine) methacrylate and N,N-dimethylaminoethylmethacrylate, quaternized with dimethyl sulphate), polyquaternium-46 (terpolymer of vinylcaprolactam, vinylpyrrolidone, and quaternized vinylimidazole), polyquaternium-47 (terpolymer of acrylic acid, methacrylamidopropyl trimethylammonium chloride, and methyl acrylate), and/or polyquaternium-67.

In certain embodiments, the hair treatment compositions of the instant disclosure include one or more cationic polymers selected from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful. A particularly preferred and useful cationic polymer is polyquaternium-10.

In certain embodiments, the hair treatment compositions include one or more cationic polymers chosen from cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, and mixtures thereof.

The hair treatment compositions according to the instant disclosure typically include about 0.01 to about 5 wt. % of one or more cationic conditioning polymers, based on the total weight of the hair treatment composition. The hair treatment compositions may include about 0.01 to about 4 wt. %, about 0.01 to about 3 wt. %, about 0.01 to about 2 wt. %, about 0.05 to about 5 wt. %, about 0.05 to about 4 wt. %, about 0.05 to about 3 wt. %, about 0.05 to about 2 wt. %, about 0.1 to about 5 wt. %, about 0.1 to about 4 wt. %, about 0.1 to about 3 wt. %, about 0.1 to about 2 wt. %, about 0.1 to about 1.5 wt. %, about 0.2 to about 5 wt. %, about 0.2 to about 4 wt. %, about 0.2 to about 3 wt. %, about 0.2 to about 2 wt. %, about 0.2 to about 1.5 wt. % of the one or more cationic polymers, based on the total weight of the hair treatment composition.

### (j) Miscellaneous Ingredients

The compositions optionally include or exclude (or are essentially free from) one or more miscellaneous ingredients. Miscellaneous ingredients are ingredients that are compatible with the compositions and do not disrupt or materially affect the basic and novel properties of the compositions. Nonlimiting examples of ingredients include preservatives, fragrances, pH adjusters, salts, chelating agents, buffers, antioxidants, flavonoids, vitamins, botanical extracts, UV filtering agents, emollients, proteins, protein hydrolysates, protein isolates, fillers (e.g., organic and/or inorganic fillers such as talc, calcium carbonate, silica, etc.) composition colorants, etc. In various embodiments, the miscellaneous ingredients are selected from preservatives, fragrances, pH adjusters, emollients, salts, chelating agents, buffers, composition colorants, and mixtures thereof.

In the context of the instant disclosure, a “composition colorant” is a compound that colors the composition but does not have an appreciable coloring effect on hair. In other words, the composition colorant is included to provide color to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the compositions, if present, will vary. Nonetheless, in various embodiments, the compositions include about 0.1 to about 15 wt. % of the one or more miscellaneous ingredients, based on the total weight of the compositions. In further embodiments, the compositions include about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. %, based on the total weight of the compositions.

pH

The pH of the hair treatment compositions will vary but is typically from about 3 to about 8. In various embodiments, the hair treatment composition has a pH of from about 3.5 to about 8, from about 4 to about 8, from about 4.5 to about 8, from about 5 to about 8, from about 5.5 to about 8, from about 6 to about 8, from about 3 to about 7.5, from about 3.5 to about 7.5, from about 4 to about 7.5, from about 4.5 to about 7.5, from about 5 to about 7.5, from about 5.5 to about 7.5, from about 6 to about 7.5, from about 4 to about 7, from about 4.5 to about 7, from about 5 to about 7, from about 5.5 to about 7, from about 6 to about 7. In further embodiments, however, it is preferable that the pH is from about 3 to less than 7, i.e., it is preferable that the composition have an acidic from about 3 to less than 7, from about 3.5 to less than 7, from about 4 to less than 7, from about 4.5 to less than 7, from about 5 to less than 7, from about 5.5 to less than 7, from about 6 to less than 7.

### Viscosity

The viscosity of the hair treatment compositions will vary, but is typically from about 1,000 to about 100,000 mPaS (cps) at 25° C. In various embodiments, the hair treatment composition has a viscosity of about 3,000 to about 100,000 mPaS, about 3,000 to about 100,000 mPaS, about 5,000 to about 100,000 mPaS, about 8,000 to about 100,000 mPaS, about 10,000 to about 100,000 mPaS, about 1,000 to about 80,000 mPas, about 3,000 to about 80,000 mPas, about 5,000 to about 80,000 mPas, about 8,000 to about 80,000 mPas, 10,000 to about 80,000 mPas, or about 10,000 to about 50,000 mPas at 25° C. The viscosities are measured using a rheometer (DHR-2, TA instruments, New Castle, DE, USA) and 40 mm parallel plate geometry. The 1 mm gap between the parallel plates is chosen. All tests carried out at 25° C. and atmospheric pressure. The sample is subjected to shear ramp starting from 0.1 1/s to 1000 1/s within a 300 second period. Sample viscosity is recorded @ shear rate of (1/s).

### Forms

The compositions of the present disclosure typically have a cream-like or gel-like like consistency, consistent with the viscosities described above. Furthermore, the composition are preferably oil-in-water emulsions. In various embodiments, the hair treatment compositions have a translucent/whitish appearance.

The hair treatment compositions may be used as a leave-in product or a rinse-off (or “rinse-out”) product.

A “leave-on” product refers to a composition that is not rinsed and/or washed from the hair after or during application of the composition onto the hair. The composition remains on the hair during drying and/or throughout styling.

A “rinse-off” product refers to a composition that is rinsed and/or washed from the hair with water either after or during the application of the composition onto the hair, and before drying and/or styling the hair. At least a portion, and typically a majority, of the composition is removed from the hair during the rinsing and/or washing.

### Optional Exclusions

All components that are positively set forth in the present disclosure may be negatively excluded from the claims, e.g., a claimed composition may be “free” or “essentially free” (“substantially free”) from one or more components that are positively set forth herein.

In a preferred embodiment, the hair treatment composition is free or essentially free from silicones. “Silicones” are also called “polysiloxanes” and refer to a class of synthetic polymers that are based on a framework of alternating silicon and oxygen (siloxane) bonds with at least one organic group attached to the silicon atom via a direct carbon-silicon bond. Nonlimiting examples of silicones include dimethicone, dimethiconol, amodimethicone, cyclomethicone, trimethicone, ceteraryl methicone. stearyl dimethicone, and the like.

In a preferred embodiment, the hair treatment composition is free or essentially free from ethanol, isopropanol, or a combination thereof. If a further preferred embodiment, the hair treatment composition is free or essentially free from C2-C6 monoalcohols, except capryl alcohol (1-hexanol). Capryl alcohol can be useful as a preservative in hair treatment compositions. In further embodiments, the hair treatment composition may be free or essentially free from C2-C6 monoalcohols including capryrl alcohol (1-hexanol).

In a preferred embodiment, the hair treatment composition is free or essentially free from anionic surfactants. Nonlimiting examples of anionic surfactants include sulfate-based surfactants (e.g., sodium laureth sulfate and sodium lauryl sulfate), acyl isethionates, acyl amino acids (such as acyl taurates, acyl glycinates, acyl glutamates, and acyl sarcosinates), alkyl sulfonates, alkyl sulfosuccinates, alkyl sulfoacetates, alkoxylated monoacids, salts thereof, and a mixtures thereof.

In a preferred embodiment, the hair treatment composition is free or essentially free from amphoteric surfactants. Nonlimiting examples of amphoteric surfactants include alkyl amphoproprionates, betaines, alkyl sultaines, alkyl amphoacetates, and combinations thereof. Preferably, at least one of the one or more amphoteric surfactants is a betaine.

In another preferred embodiment, the hair treatment composition is free or essentially free from both anionic surfactants and amphoteric surfactants.

In various embodiments, the hair treatment composition is free or essentially free from polymers, copolymers, and crosspolymers formed with acrylate or methacrylate monomers, e.g, free or essentially free from polyacrylic acid and polyacrylate polymers and crosspolymers.

### Cationic Surfactants

Non-limiting examples of cationic surfactants include behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3) oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The cationic surfactant(s) may also be chosen from optionally polyoxyalkylenated, primary, secondary or tertiary fatty amines, or salts thereof, and quaternary ammonium salts, and combinations thereof.

In some cases it is useful to use salts such as chloride salts of the quaternary ammonium compounds.

The fatty amines generally comprise at least one C8-C30 hydrocarbon-based chain.
A. Examples of quaternary ammonium salts that may especially be mentioned include those corresponding to the general formula (I) below:

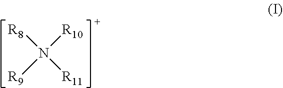

in which the groups R& to R₁₁, which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms and preferably from 12 to 24 carbon atoms. The aliphatic groups may comprise heteroatoms especially such as oxygen, nitrogen, sulfur and halogens. The aliphatic groups are chosen, for example, from C₁-C₃₀ alkyl, C₂-C₃₀ alkenyl, C₁-C₃₀ alkoxy, polyoxy (C₂-C₆)alkylene, C₁-C₃₀ alkylamide, (C₁₂-C₂₂)alkylamido (C₂-C₆)alkyl, (C₁₂-C₂₂)alkyl acetate and C₁-C₃₀ hydroxyalkyl groups; X⁻ is an anion chosen from the group of halides, phosphates, acetates, lactates, (C₁-C₄)alkyl sulfates, and (C₁-C₄)alkyl- or (C₁-C₄)alkylarylsulfonates.

Among the quaternary ammonium salts of formula (III), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethylammonium, cetyltrimethylammonium or benzyldimethylstearylammonium salts, or, on the other hand, oleocetyldimethylhydroxyethylammonium salts, palmitylamidopropyltrimethylammonium salts, stearamidopropyltrimethylammonium salts and stearamidopropyldimethylcetearylammonium salts.
B. a quaternary ammonium salt of imidazoline, such as, for example, those of formula (II) below:

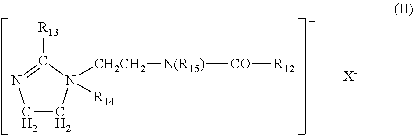

in which R₁₂ represents an alkenyl or alkyl group comprising from 8 to 30 carbon atoms, derived for example from tallow fatty acids, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, alkyl sulfates, alkyl- or alkylaryl-sulfonates in which the alkyl and aryl groups preferably comprise, respectively, from 1 to 20 carbon atoms and from 6 to 30 carbon atoms. R₁₂ and R₁₃ preferably denote a combination of alkenyl or alkyl groups containing from 12 to 21 carbon atoms, derived for example from tallow fatty acids, R₁₄ preferably denotes a methyl group, and R₁₅ preferably denotes a hydrogen atom. Such a product is sold, for example, under the name REWOQUAT W 75 by the company Rewo;
C. a quaternary diammonium or triammonium salt, in particular of formula (III):

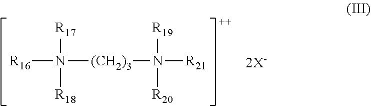

in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates. Such compounds are, for example, Finquat CT-P, sold by the company Finetex (Quaternium 89), and Finquat CT, sold by the company Finetex (Quaternium 75),
D. Cationic/cationizable surfactants, for example of the general structure

R4-A-R5-B
wherein R4 is a saturated or unsaturated, straight or branched alkyl chain with 8 to 24 carbon atoms, R5 is a straight or branched alkyl chain with 1 to 4 carbon atoms, A is selected from:

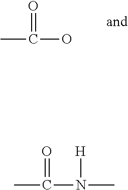

and B is selected from

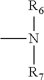

wherein R₆ and R₇ are the same or different is H or an alkyl chain with 1 to 4 C atoms, hydroxyl alkyl chain with 1 to 4 C atoms and di hydroxyl alkyl chain with 2 to 4 C atoms,

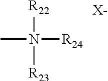

R₂₂ and R₂₃ are the same or different, an alkyl chain with 1 to 4 C atoms, hydroxyl alkyl chain with 1 to 4 C atoms and di hydroxyl alkyl chain with 2 to 4 C atoms, R₂₄ is an alkyl chain with 1 to 4 C atoms, hydroxyl alkyl chain with 1 to 4 C atoms or di hydroxyl alkyl chain with 2 to 4 C atoms.

In some instances, R₄ is saturated or unsaturated, straight or branched alkyl chain with 10 to 24 C atoms, more preferably 12 to 22 C atoms and R₅ is straight or branched alkyl group with 1 to 4 C atoms, and A, B, R₆ to R₁₀ are same as above.

Non-limiting suitable examples are stearyloxypropyl amine, palmityloxypropyl amine, stearyloxypropyldimethyl amine, stearyloxypropyldiethyl amine, stearyloxyethylyldimethyl amine, stearyloxyethyl amine, myristyloxypropyl amine, myristyloxypropyldimethyl amine, palmitamidopropyl amine, palmitamidopropyl methylamine, palmitamidopropyl diethylamine, palmitamidopropyl dibutylamine, palmitamidopropyl buylamine, palmitamidopropyl dipropylamine, palmitamidopropyl propylamine, palmitamidopropyl dihydroxyethylamine, palmitamidopropyl hydroxyethylamine, palmitamidopropyl dihydroxypropylamine, palmitamidopropyl hydroxypropylamine, lauramidopropyl amine, lauramidopropyl methylamine, lauramidopropyl diethylamine, lauramidopropyl dibutylamine, lauramidopropyl buylamine, lauramidopropyl dipropylamine, lauramidopropyl propylamine, lauramidopropyl dihydroxyethylamine, lauramidopropyl hydroxyethylamine, lauramidopropyl dihydroxypropylamine, lauramidopropyl hydroxypropylamine, stearamidopropyl amine, stearamidopropyl dimethylamine, steara midopropyl diethylamine, stearamidopropyldibutylamine, stearamidopropyl butylamine, stearamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, behenamidopropyl amine, behenamidopropyl methylamine, behenamidopropyl diethylamine, behenamidopropyl dibutylamine, behenamidopropyl butylamine, behenamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, dipalmitamidopropyl methylamine, dipalmitamidopropyl ethylamine, dipalmitamidopropyl butylamine, dipalmitamidopropyl propylamine, dipalmitamidopropyl hydroxyethylamine, dipalmitamidopropyl hydroxypropylamine, dilauramidopropyl amine, dilauramidopropyl methylamine, dilauramidopropyl buylamine, dilauramidopropyl hydroxyethylamine, dilauramidopropyl hydroxypropylamine, distearamidopropyl amine, distearamidopropyl methylamine, dibehenamidopropyl propylamine, dibehenamidopropyl hydroxyethylamine, palmitoamidopropyl trimethyl ammonium chloride, stearamidopropyl trimethylammonium chloride, behenamidopropyl tri hydroxyethalmonium chloride, distearylamidopropyl dimethyl ammonium chloride, dicetylamidodihydroxyethyl ammonium chloride, palmitoylpropyl amine, palmitoylpropyl methylamine, palmitoylpropyl diethylamine, palmitoylpropyl dibutylamine, palmitoylpropyl buylamine, palmitoylpropyl dipropylamine, palmitoylpropyl propylamine, palmitoylpropyl dihydroxyethylamine, palmitoylpropyl hydroxyethylamine, palmitoylpropyl dihydroxypropylamine, palmitoylpropyl hydroxypropylamine, myristoylpropyl amine, myristoylpropyl methylamine, myristoylpropyl diethylamine, myristoylpropyl dibutylamine, myristoylpropyl buylamine, myristoylpropyl dipropylamine, myristoylpropyl propylamine, myristoylpropyl dihydroxyethylamine, myristoylpropyl hydroxyethylamine, myristoylpropyl dihydroxypropylamine, myristoylpropyl hydroxypropylamine, stearoylpropyl amine, stearoylpropyl methylamine, stearoylpropyl diethylamine, stearoylpropyl dibutylamine, stearoylpropyl butylamine, stearoylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, behenylpropyl amine, behenylpropyl methylamine, behenylpropyl diethylamine, behenylpropyl dibutylamine, behenylpropyl butylamine, behenylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, dipalmitoylpropyl methylamine, dipalmitoylpropyl ethylamine, dipalmitylpropyl butylamine, dipalmitylpropyl propylamine, dipalmitylpropyl hydroxyethylamine, dipalmitylpropyl hydroxypropylamine, dilauroylpropyl amine, dilauroylpropyl methylamine, dilauroylpropyl buylamine, dilauroylpropyl hydroxyethylamine, dilauroylpropyl hydroxypropylamine, distearylpropyl amine, distearylpropyl methylamine, dibehenylpropyl propylamine, dibehenylpropyl hydroxyethylamine, palmitylpropyl trimethyl ammonium chloride, stearylpropyl trimethylammonium chloride, behenylpropyl tri hydroxyethalmonium chloride, distearylpropyl dimethyl ammonium chloride, dicetyldihydroxyethyl ammonium chloride, dioleoylethylhydroxyethylmonium methosulfate, and dicocoylethylhydroxyethylmonium methosulfate.

Cationizable surfactants or amphiphilic surfactants may be chosen from fatty alkylamines, preferably, fatty dialkylamines. In some cases, the fatty dialkylamines may be fatty dimethylamines. Non-limiting examples include dimethyl lauramine, dimethyl behenamine, dimethyl cocamine, dimethyl myristamine, dimethyl palmitamine, dimethyl stearamine, dimethyl tallowamine, dimethyl soyamine, and combinations thereof. In various embodiments, it is preferably that at least one of the one or more cationic surfactants is a fatty dialkylamine.

Fatty dialkylamines include fatty amidoamine compounds, their salts, and combinations thereof. Non-limiting examples include oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, and palmitamidopropyl dimethylamine. In a preferred embodiment, the compositions of the instant disclosure include stearamidopropyl dimethylamine, and optionally one or more additional cationic surfactants

Non-polymeric, mono-, di-, and/or tri-carboxylic acids may be used to “neutralize” the fatty dialkylamines. In some cases, the one or more non-polymeric, mono-, di-, and/or tri-carboxylic acids include at least one dicarboxylic acid. Non-limiting examples include lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, benzoic acid, and combinations thereof. In particular, lactic acid or tartaric acid or combinations thereof are useful, especially in combination with fatty dimethylamines such as, for example, stearamidopropyl dimethylamine.

### PREFERRED EMBODIMENTS

In a preferred embodiment, the hair treatment composition comprises, consists essentially of, or consists of:
(a) about 0.1 to about 5 wt. %, preferably about 0.5 to about 4 wt. %, more preferably about 1 to about 3 wt. % of one or more cationic surfactants, wherein preferably, the one or more cationic surfactants are selected from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, salts thereof, or mixtures thereof, wherein preferably the one or more cationic surfactants is selected from cetrimonium chloride, behentrimonium chloride, brassicamidopropyl dimethylamine, stearamidopropyl dimethylamine, salts thereof, or mixtures thereof;
(b) about 1 to about 10 wt. %, preferably about 2 to about 9 wt. %, more preferably about 3 to about 8 wt. % of one or more solid fatty alcohols, wherein preferably the one or more solid fatty alcohols are selected from stearyl alcohol, cetyl alcohol, cetearyl alcohol, myristyl alcohol, behenyl alcohol, or mixtures thereof;
(c) about 0.1 to about 6 wt. %, preferably about 0.2 to about 5 wt. %, more preferably about 0.5 to about 4 wt. % of one or more liquid fatty alcohols, wherein preferably the one or more liquid fatty alcohols are selected from isostearyl alcohol, oleyl alcohol, octyldodecanol, linoleyl alcohol, linolenyl alcohol, lauryl alcohol, 2-hexyldecanol, 2-heptyldecanol, enanthic alcohol, caprylic alcohol, pelargonic alcohol, capric alcohol, or mixtures thereof;
(d) about 0.3 to about 8 wt. %, preferably about 0.5 to about 6 wt. %, more preferably about 1 to about 5 wt. % of one or more low polar oils having a Polarity Index of about 23 mN/m to about 40 mN/m, wherein preferably the one or more low polar oils is a non-silicone oil selected from fatty carbonates, fatty ethers, liquid linear or branched carboxylic acid esters, or mixtures thereof, wherein more preferably the one or more non-silicone, low polar oils are selected from stearyl ether, dicaprylyl ether, dicetyl ether distearyl ether, dodecyl ether, dilauryl ether, dimyristyl ether, diisononyl ether, dicaprylyl carbonate, diethyl carbonate, dihexyl carbonate, diethylhexyl carbonate, dimethoxyphenyl phenyloxoethyl ethylcarbonate, dimethyl carbonate, dipropyl carbonate, dipropylheptyl carbonate, dioctyl carbonate, or mixtures thereof;
   wherein a weight ratio of a combination of (c) and (d) to a combination of (a) and (b) is from about 0.5:1 to about 3:1, preferably from about 0.5:1 to about 2:1, more preferably about 0.5:1 to about 1.5:1 (((c)+(d)):((a)+(b))); and
(e) about 60 to about 92 wt. %, preferably about 70 to about 90 wt. %, more preferably about 75 to 90 wt. % of water;
(f) optionally, about 0.01 to about 15 wt. %, preferably about 0.05 to about 10 wt. %, more preferably about 0.1 to about 5 wt. % of one or more water soluble solvents selected from C₂-C₆ monoalcohols, glycerin, polyols, glycols, glycol ethers, and mixtures thereof, wherein preferably, the one of the one or more water soluble solvents are selected from glycerin and glycols, for example, glycols selected from glycerin, propylene glycol, butylene glycol, pentylene glycol, hexylene glycol, caprylyl glycol, dipropylene glycol, or mixtures thereof, wherein optionally the compositions is free or essentially free from ethanol and isopropyl alcohol;
(g) optionally, about 0.01 to about 5 wt. %, preferably about 0.05 to about 3 wt. %, more preferably about 0.1 to about 2 wt. % of one or more mono-, di-, and/or tri-carboxylic acids, preferably wherein at least one of the one or more one or more mono-, di-, and/or tri-carboxylic acids are selected from lactic acid, tartaric acid, citric acid, or mixtures thereof;
(h) optionally, about 0.01 to about 15 wt. %, preferably about 0.05 to about 10 wt. %, more preferably about 0.1 to about 5 wt. % of one or more fatty compounds other than (b), (c), and (d), for example, one or more fatty compounds other than (b), (c), and (d) selected from oils, waxes, alkanes, fatty acids, fatty esters, triglyceride compounds, lanolin, hydrocarbons, derivatives thereof, silicones, and mixtures thereof, wherein preferably, the composition is free or essentially free of silicones;
(i) optionally, about 0.01 to about 5 wt. %, preferably about 0.05 to about 4 wt. %, more preferably about 0.1 to about 3 wt. % of one or more cationic conditioning polymers;
(j) optionally, about 0.1 to about 10 wt. %, preferably about 0.5 to about 8 wt. %, more preferably about 1 to about 5 wt. % of one or more miscellaneous ingredients, preferably wherein the one or more miscellaneous ingredients are selected from preservatives, fragrances, pH adjusters, salts, chelating agents, buffers, antioxidants, flavonoids, vitamins, botanical extracts, UV filtering agents, emollients, proteins, protein hydrolysates, protein isolates, fillers (e.g., organic and/or inorganic fillers such as talc, calcium carbonate, silica, etc.) composition colorants, or mixtures thereof, more preferably, wherein the one or more miscellaneous ingredients are selected from preservatives, fragrances, pH adjusters, emollients, salts, chelating agents, buffers, composition colorants, and mixtures thereof.
   wherein all percentages by weight are based on a total weight of the composition.

Preferably, the hair treatment composition described above is an oil-in-water emulsion.

The hair treatment composition described above preferably has a pH of from about 3 to about 8. Even more preferably, the hair treatment composition described above has a pH of from about 5 to about 7.5.

The hair treatment composition described above preferably has a viscosity of about 1,000 to about 100,000 mPaS (cps) at 25° C. Even more preferably, the hair treatment composition described above has a viscosity of about 5,000 to about 80,000 mPaS (cps) at 25° C. The viscosities of the formulations are measured using a rheometer (DHR-2, TA instruments, New Castle, DE, USA) and 40 mm parallel plate geometry. The 1 mm gap between the parallel plates is chosen. All tests carried out at 25° C. and atmospheric pressure. The sample is subjected to shear ramp starting from 0.1 1/s to 1000 1/s within a 300 second period. Sample viscosity is recorded @ shear rate of (1/s).

In a preferred embodiment, the hair treatment composition comprises, consists essentially of, or consists of:
(a) one or more cationic surfactants, preferably one or more cationic surfactants selected from preferably, the one or more cationic surfactants are selected from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, salts thereof, or mixtures thereof, wherein preferably the one or more cationic surfactants is selected from cetrimonium chloride, behentrimonium chloride, brassicamidopropyl dimethylamine, stearamidopropyl dimethylamine, salts thereof, or mixtures thereof;
(b) one or more solid fatty alcohols selected from stearyl alcohol, cetyl alcohol, cetearyl alcohol, myristyl alcohol, behenyl alcohol, or mixtures thereof;
   wherein (a) and (b) are in a combined amount of about 2 to about 15 wt. %, preferably in a combined amount of about of about 3 to about 12 wt. %, even more preferably in a combined amount of about 5 to about 10 wt. %
(c) one or more liquid fatty alcohols selected from isostearyl alcohol, oleyl alcohol, octyldodecanol, linoleyl alcohol, linolenyl alcohol, lauryl alcohol, 2-hexyldecanol, 2-heptyldecanol, enanthic alcohol, caprylic alcohol, pelargonic alcohol, capric alcohol, or mixtures thereof;
(d) one or more low polar oils having a Polarity Index of about 23 mN/m to about 40 mN/m, wherein preferably the one or more low polar oils are non-silicone oils selected from fatty carbonates, fatty ethers, liquid linear or branched carboxylic acid esters, or mixtures thereof, wherein more preferably the one or more non-silicone, low polar oils are selected from stearyl ether, dicaprylyl ether, dicetyl ether distearyl ether, dodecyl ether, dilauryl ether, dimyristyl ether, diisononyl ether, dicaprylyl carbonate, diethyl carbonate, dihexyl carbonate, diethylhexyl carbonate, dimethoxyphenyl phenyloxoethyl ethylcarbonate, dimethyl carbonate, dipropyl carbonate, dipropylheptyl carbonate, dioctyl carbonate, or mixtures thereof
   wherein (c) and (d) are in a combined amount of about 2 to about 15 wt. %, preferably in a combined amount of about 3 to about 10 wt. %, more preferably a combined amount of about 4 to about 7 wt. %;
   and a weight ratio of a combination of (c) and (d) to a combination of (a) and (b) is from about 0.5:1 to about 3:1, preferably from about 0.5:1 to about 2:1, more preferably about 0.5:1 to about 1.5:1 (((c)+(d)):((a)+(b)))
(e) about 60 to about 92 wt. %, preferably about 70 to about 90 wt. %, more preferably about 75 to 90 wt. % of water;
(f) optionally, about 0.01 to about 15 wt. %, preferably about 0.05 to about 10 wt. %, more preferably about 0.1 to about 5 wt. % of one or more water soluble solvents selected from C₂-C₆ monoalcohols, glycerin, polyols, glycols, glycol ethers, and mixtures thereof, wherein preferably, the one of the one or more water soluble solvents are selected from glycerin and glycols, for example, glycols selected from glycerin, propylene glycol, butylene glycol, pentylene glycol, hexylene glycol, caprylyl glycol, dipropylene glycol, or mixtures thereof, wherein optionally the compositions is free or essentially free from ethanol and isopropyl alcohol;
(g) optionally, about 0.01 to about 5 wt. %, preferably about 0.05 to about 3 wt. %, more preferably about 0.1 to about 2 wt. % of one or more mono-, di-, and/or tri-carboxylic acids, preferably wherein at least one of the one or more one or more mono-, di-, and/or tri-carboxylic acids are selected from lactic acid, tartaric acid, citric acid, or mixtures thereof;
(h) optionally, about 0.01 to about 15 wt. %, preferably about 0.05 to about 10 wt. %, more preferably about 0.1 to about 5 wt. % of one or more fatty compounds other than (b), (c), and (d), for example, one or more fatty compounds other than (b), (c), and (d) selected from oils, waxes, alkanes, fatty acids, fatty esters, triglyceride compounds, lanolin, hydrocarbons, derivatives thereof, silicones, and mixtures thereof, wherein preferably, the composition is free or essentially free of silicones;
(i) optionally, about 0.01 to about 5 wt. %, preferably about 0.05 to about 4 wt. %, more preferably about 0.1 to about 3 wt. % of one or more cationic conditioning polymers;
(j) optionally, about 0.1 to about 10 wt. %, preferably about 0.5 to about 8 wt. %, more preferably about 1 to about 5 wt. % of one or more miscellaneous ingredients, preferably wherein the one or more miscellaneous ingredients are selected from preservatives, fragrances, pH adjusters, salts, chelating agents, buffers, antioxidants, flavonoids, vitamins, botanical extracts, UV filtering agents, emollients, proteins, protein hydrolysates, protein isolates, fillers (e.g., organic and/or inorganic fillers such as talc, calcium carbonate, silica, etc.) composition colorants, or mixtures thereof, more preferably, wherein the one or more miscellaneous ingredients are selected from preservatives, fragrances, pH adjusters, emollients, salts, chelating agents, buffers, composition colorants, and mixtures thereof.
   wherein all percentages by weight are based on a total weight of the composition.

Preferably, the hair treatment composition described above is an oil-in-water emulsion.

The hair treatment composition described above preferably has a pH of from about 3 to about 8. Even more preferably, the hair treatment composition described above has a pH of from about 5 to about 7.5.

The hair treatment composition described above preferably has a viscosity of about 1,000 to about 100,000 mPaS (cps) at 25° C. Even more preferably, the hair treatment composition described above has a viscosity of about 5,000 to about 80,000 mPaS (cps) at 25° C. The viscosities of the formulations are measured using a rheometer (DHR-2, TA instruments, New Castle, DE, USA) and 40 mm parallel plate geometry. The 1 mm gap between the parallel plates is chosen. All tests carried out at 25° C. and atmospheric pressure. The sample is subjected to shear ramp starting from 0.1 1/s to 1000 1/s within a 300 second period. Sample viscosity is recorded @ shear rate of (1/s).

In a preferred embodiment, the hair treatment composition comprises, consists essentially of, or consists of:
(a) about 0.1 to about 5 wt. %, preferably about 0.5 to about 4 wt. %, more preferably about 1 to about 3 wt. % of one or more cationic surfactants, wherein preferably, the one or more cationic surfactants are selected from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, salts thereof, or mixtures thereof, wherein preferably the one or more cationic surfactants is selected from cetrimonium chloride, behentrimonium chloride, brassicamidopropyl dimethylamine, stearamidopropyl dimethylamine, salts thereof, or mixtures thereof;
(b) about 1 to about 10 wt. %, preferably about 2 to about 9 wt. %, more preferably about 3 to about 8 wt. % of one or more solid fatty alcohols, wherein preferably the one or more solid fatty alcohols are selected from stearyl alcohol, cetyl alcohol, cetearyl alcohol, myristyl alcohol, behenyl alcohol, or mixtures thereof;
   wherein (a) and (b) are in a combined amount of about 2 to about 15 wt. %, preferably in a combined amount of about of about 3 to about 12 wt. %, even more preferably in a combined amount of about 5 to about 10 wt. %
(c) about 0.1 to about 6 wt. %, preferably about 0.2 to about 5 wt. %, more preferably about 0.5 to about 4 wt. % of one or more liquid fatty alcohols, wherein preferably the one or more liquid fatty alcohols are selected from isostearyl alcohol, oleyl alcohol, octyldodecanol, linoleyl alcohol, linolenyl alcohol, lauryl alcohol, 2-hexyldecanol, 2-heptyldecanol, enanthic alcohol, caprylic alcohol, pelargonic alcohol, capric alcohol, or mixtures thereof;
(d) about 0.3 to about 8 wt. %, preferably about 0.5 to about 6 wt. %, more preferably about 1 to about 5 wt. % of one or more low polar oils having a Polarity Index of about 23 mN/m to about 40 mN/m, wherein preferably the one or more low polar oils are non-silicone oils selected from fatty carbonates, fatty ethers, liquid linear or branched carboxylic acid esters, or mixtures thereof, wherein more preferably the one or more non-silicone, low polar oils are selected from stearyl ether, dicaprylyl ether, dicetyl ether distearyl ether, dodecyl ether, dilauryl ether, dimyristyl ether, diisononyl ether, dicaprylyl carbonate, diethyl carbonate, dihexyl carbonate, diethylhexyl carbonate, dimethoxyphenyl phenyloxoethyl ethylcarbonate, dimethyl carbonate, dipropyl carbonate, dipropylheptyl carbonate, dioctyl carbonate, or mixtures thereof;
   wherein (c) and (d) are in a combined amount of about 2 to about 15 wt. %, preferably in a combined amount of about 3 to about 10 wt. %, more preferably a combined amount of about 4 to about 7 wt. %; and
   wherein a weight ratio of a combination of (c) and (d) to a combination of (a) and (b) is from about 0.5:1 to about 3:1, preferably from about 0.5:1 to about 2:1, more preferably about 0.5:1 to about 1.5:1 (((c)+(d)):((a)+(b))); and
(e) about 60 to about 92 wt. %, preferably about 70 to about 90 wt. %, more preferably about 75 to 90 wt. % of water;
(f) optionally, about 0.01 to about 15 wt. %, preferably about 0.05 to about 10 wt. %, more preferably about 0.1 to about 5 wt. % of one or more water soluble solvents selected from C₂-C₆ monoalcohols, glycerin, polyols, glycols, glycol ethers, and mixtures thereof, wherein preferably, the one of the one or more water soluble solvents are selected from glycerin and glycols, for example, glycols selected from glycerin, propylene glycol, butylene glycol, pentylene glycol, hexylene glycol, caprylyl glycol, dipropylene glycol, or mixtures thereof, wherein optionally the compositions is free or essentially free from ethanol and isopropyl alcohol;
(g) optionally, about 0.01 to about 5 wt. %, preferably about 0.05 to about 3 wt. %, more preferably about 0.1 to about 2 wt. % of one or more mono-, di-, and/or tri-carboxylic acids, preferably wherein at least one of the one or more one or more mono-, di-, and/or tri-carboxylic acids are selected from lactic acid, tartaric acid, citric acid, or mixtures thereof;
(h) optionally, about 0.01 to about 15 wt. %, preferably about 0.05 to about 10 wt. %, more preferably about 0.1 to about 5 wt. % of one or more fatty compounds other than (b), (c), and (d), for example, one or more fatty compounds other than (b), (c), and (d) selected from oils, waxes, alkanes, fatty acids, fatty esters, triglyceride compounds, lanolin, hydrocarbons, derivatives thereof, silicones, and mixtures thereof, wherein preferably, the composition is free or essentially free of silicones;
(i) optionally, about 0.01 to about 5 wt. %, preferably about 0.05 to about 4 wt. %, more preferably about 0.1 to about 3 wt. % of one or more cationic conditioning polymers;
(j) optionally, about 0.1 to about 10 wt. %, preferably about 0.5 to about 8 wt. %, more preferably about 1 to about 5 wt. % of one or more miscellaneous ingredients, preferably wherein the one or more miscellaneous ingredients are selected from preservatives, fragrances, pH adjusters, salts, chelating agents, buffers, antioxidants, flavonoids, vitamins, botanical extracts, UV filtering agents, emollients, proteins, protein hydrolysates, protein isolates, fillers (e.g., organic and/or inorganic fillers such as talc, calcium carbonate, silica, etc.) composition colorants, or mixtures thereof, more preferably, wherein the one or more miscellaneous ingredients are selected from preservatives, fragrances, pH adjusters, emollients, salts, chelating agents, buffers, composition colorants, and mixtures thereof.
   wherein all percentages by weight are based on a total weight of the composition.

Preferably, the hair treatment composition described above is an oil-in-water emulsion.

The hair treatment composition described above preferably has a pH of from about 3 to about 8. Even more preferably, the hair treatment composition described above has a pH of from about 5 to about 7.5.

The hair treatment composition described above preferably has a viscosity of about 1,000 to about 100,000 mPaS (cps) at 25° C. Even more preferably, the hair treatment composition described above has a viscosity of about 5,000 to about 80,000 mPaS (cps) at 25° C. The viscosities of the formulations are measured using a rheometer (DHR-2, TA instruments, New Castle, DE, USA) and 40 mm parallel plate geometry. The 1 mm gap between the parallel plates is chosen. All tests carried out at 25° C. and atmospheric pressure. The sample is subjected to shear ramp starting from 0.1 1/s to 1000 1/s within a 300 second period. Sample viscosity is recorded @ shear rate of (1/s).

### EXAMPLES

Various changes can be made in the above-described compositions and methods without departing from the scope of the invention. Accordingly, it is intended that all disclosure contained in the above description and in the examples given below, shall be interpreted as illustrative and not in a limiting sense.

### Example 1

### (Inventive Composition)

Compositions A through G shown in Table 1A and Table 1B were prepared by combining water and organic acids and heating to 70° C. Upon reaching 70° C., cationic surfactants, solid fatty alcohols, liquid fatty alcohols, and low polarity oils were added and emulsified for 10-15 minutes. The compositions were cooled to below 50° C. before miscellaneous ingredients, such as preservatives and fragrances were added. The compositions were continuously homogenized and cooled to room temperature (about 25° C.).

TABLE 1A

| Composition | | | A | B | C | D | E | F | G |
|---|---|---|---|---|---|---|---|---|---|
| (a) | Cationic | BRASSICAMIDOPROPYL | 1.3 | 1.3 | 1.3 | 1.3 | 1.3 | 1.3 | 1.3 |
| | Surfactant | DIMETHYLAMINE | | | | | | | |
| | | STEARAMIDOPROPYL | | | | | | | |
| | | DIMETHYLAMINE | | | | | | | |
| (b) | Solid | MYRISTYL ALCOHOL | 2.6 | 2.6 | 2 | 2 | 2.6 | 2.6 | 2.2 |
| | Fatty | CETEARYL ALCOHOL | 3.3 | 3.3 | 3.3 | 3.3 | 3.3 | 3.3 | 3.3 |
| | Alcohol | CETYL ALCOHOL | 0.2 | 0.2 | | | 0.2 | 0.2 | 0.1 |
| Total (a) + (b) | | | 7.4 | 7.4 | 6.6 | 6.6 | 7.4 | 7.4 | 6.9 |
| (c) | Liquid | LAURYL ALCOHOL | 1.9 | 1.9 | | | 1.9 | 1.9 | 0.7 |
| | Fatty | OLEYL ALCOHOL | | | 2.8 | | | | |
| | Alcohol | OCTYLDODECANOL | | | | 2.8 | | | |
| (d) | Low Polar | DICAPRYLYL | | 2.7 | | | | | |
| | Oil | CARBONATE | | | | | | | |
| | | DICAPRYLYL ETHER | 2.7 | | 2.7 | 2.7 | 2.7 | 2.7 | 3.5 |
| Total (c) + (d) | | | 4.6 | 4.6 | 5.5 | 5.5 | 4.6 | 4.6 | 4.2 |
| Ratio ((c) + (d)):((a) + (b)) | | | 0.6:1 | 0.6:1 | 0.8:1 | 0.8:1 | 0.6:1 | 0.6:1 | 0.6:1 |
| | Water | CAPRYLYL GLYCOL | 0.3 | 0.3 | 0.3 | 0.3 | 0.3 | 0.3 | 0.3 |
| | Soluble | BUTYLENE GLYCOL | | | | | 2.0 | | |
| | Solvent | PROPYLENE GLYCOL | | | | | | 2.0 | |
| | Organic | LACTIC ACID | | | | | 0.2 | 0.2 | 0.2 |
| | Acid | TARTARIC ACID | 0.2 | 0.2 | 0.2 | 0.2 | | | |

| | | | | | | | |
|---|---|---|---|---|---|---|---|
| Miscellaneous ¹ | ≤5 | ≤5 | ≤5 | ≤5 | ≤5 | ≤5 | ≤5 |

| | | |
|---|---|---|
| Water | WATER | Q.S. |

| pH | 6-7.5 | | | | | | |
|---|---|---|---|---|---|---|---|
| Viscosity | 10,000-40,000 mPaS at 25° C. | | | | | | |
| Stable | Y | Y | Y | Y | Y | Y | Y |

¹ Preservatives, fragrances, pH adjusters, salts, chelating agents, buffers, antioxidants, flavonoids, vitamins, botanical extracts, emollients, UV filtering agents, proteins, protein hydrolysates, protein isolates, fillers, composition colorants, or mixtures thereof.

TABLE 1B

| Composition | | | H | I | J |
|---|---|---|---|---|---|
| (a) | Cationic | BRASSICAMIDOPROPYL | 1.3 | 1.1 | |
| | Surfactant | DIMETHYLAMINE | | | |
| | | STEARAMIDOPROPYL | | | 1.3 |
| | | DIMETHYLAMINE | | | |
| (b) | Solid | MYRISTYL ALCOHOL | 2.2 | 1.9 | 2.6 |
| | Fatty | CETEARYL ALCOHOL | 3.3 | 2.8 | 3.3 |
| | Alcohol | CETYL ALCOHOL | 0.1 | 0.1 | 0.2 |
| Total (a) + (b) | | | 6.9 | 5.9 | 7.4 |
| (c) | Liquid | LAURYL ALCOHOL | 0.7 | 0.7 | 1.9 |
| | Fatty | OLEYL ALCOHOL | | | |
| | Alcohol | OCTYLDODECANOL | | | |
| (d) | Low Polar | DICAPRYLYL | 3.5 | 3.5 | |
| | Oil | CARBONATE | | | |
| | | DICAPRYLYL ETHER | | | 2.7 |
| Total (c) + (d) | | | 4.2 | 4.2 | 4.6 |
| Ratio ((c) + (d)):((a) + (b)) | | | 0.6:1 | 0.7:1 | 0.6:1 |
| (f) | Water | CAPRYLYL GLYCOL | 0.3 | 0.3 | 0.3 |
| | Soluble | BUTYLENE GLYCOL | | 1.0 | |
| | Solvent | PROPYLENE GLYCOL | | | |
| (g) | Organic | LACTIC ACID | 0.2 | 0.2 | |
| | Acid | TARTARIC ACID | | | 0.2 |

| | | | |
|---|---|---|---|
| Miscellaneous ¹ | ≤5 | ≤5 | ≤5 |

| | | | |
|---|---|---|---|
| (e) | Water | WATER | Q.S. |

| pH | 6-7.5 | | |
|---|---|---|---|
| Viscosity | 10,000-40,000 mPaS | | |
| | at 25° C. | | |
| Stable | Y | Y | Y |

¹ Preservatives, fragrances, pH adjusters, salts, chelating agents, buffers, antioxidants, flavonoids, vitamins, botanical extracts, emollients, UV filtering agents, proteins, protein hydrolysates, protein isolates, fillers, composition colorants, or mixtures thereof.

### Example 2

### (Stability and Performance Testing)

Stability and performance testing was carried out on the compositions set forth in Table 2, below. Inventive composition A in Table 2 is the same as inventive composition A in Table 1A of Example 1. Comparative compositions C1-C4 were useful for determining the criticality of the cationic surfactant (a), the solid fatty alcohol (b), the liquid fatty alcohol (c), and the low polar oil (d). Comparative composition C1 is identical to inventive composition A except it lacks a cationic surfactant (a). Comparative composition C2 is identical to inventive composition A except it lacks a solid fatty alcohol (b). Comparative composition C3 is identical to inventive composition A except it lacks a liquid fatty alcohol (c). Comparative composition C4 is identical to inventive composition A except it lacks a low polar oil. Water replaced the missing ingredients.

TABLE 2

| | | | Inventive | Comparative | | | |
|---|---|---|---|---|---|---|---|
| Composition | | | A | C1 | C2 | C3 | C4 |
| (a) | Cationic | BRASSICAMIDOPROPYL | 1.3 | | 1.3 | 1.3 | 1.3 |
| | Surfactant | DIMETHYLAMINE | | | | | |
| (b) | Solid Fatty | MYRISTYL ALCOHOL | 2.6 | 2.6 | | 2.6 | 2.6 |
| | Alcohol | CETEARYL ALCOHOL | 3.3 | 3.3 | | 3.3 | 3.3 |
| | | CETYL ALCOHOL | 0.2 | 0.2 | | 0.2 | 0.2 |
| Total (a) + (b) | | | 7.4 | 6.1 | 1.3 | 7.4 | 7.4 |
| (c) | Liquid F.A. | LAURYL ALCOHOL | 1.9 | 1.9 | 1.9 | | 1.9 |
| (d) | L.P. Oil | DICAPRYLYL ETHER | 2.7 | 2.7 | 2.7 | 2.7 | |
| Total (c) + (d) | | | 4.6 | 4.6 | 4.6 | 2.7 | 1.9 |
| Ratio ((c) + (d)):((a) + (b)) | | | 0.6:1 | 0.8:1 | 3.5:1 | 0.4:1 | 0.3:1 |
| (f) | W.S. | CAPRYLYL GLYCOL | 0.3 | 0.3 | 0.3 | 0.3 | 0.3 |
| | Solvent | | | | | | |
| (g) | Organic | TARTARIC ACID | 0.2 | 0.2 | 0.2 | 0.2 | 0.2 |
| | Acid | | | | | | |

| | | | | | |
|---|---|---|---|---|---|
| Miscellaneous ¹ | ≤5 | ≤5 | ≤5 | ≤5 | ≤5 |

| | | | |
|---|---|---|---|
| (e) | Water | WATER | Q.S. |

| pH | 6-7.5 | 2.5 ² | 6-7.5 | | |
|---|---|---|---|---|---|
| Stable | Y | N | N | Y | Y |
| Lack or Residue and Flaking | Y | NA | NA | N | N |

¹ Preservatives, fragrances, pH adjusters, salts, chelating agents, buffers, antioxidants, flavonoids, vitamins, botanical extracts, emollients, UV filtering agents, proteins, protein hydrolysates, protein isolates, fillers, composition colorants, or mixtures thereof.
² The pH is much lower because the added organic acids are not being neutralized by the fatty amine (brassicamidopropyl dimethylamine).

All compositions of Table 2 were processed in the same manner. Water and organic acids were combined and heating to 70° C. Upon reaching 70° C., cationic surfactants, solid fatty alcohols, liquid fatty alcohols, and low polarity oils were added and emulsified for 10-15 minutes. The compositions were cooled to below 50° C. before miscellaneous ingredients, such as preservatives and fragrances were added. The compositions were continuously homogenized and cooled to room temperature (about 25° C.).

The compositions were placed in transparent glass beakers and visually monitored over a 24-hour period while being maintained at room temperature (about 25° C.). Comparative compositions C1 and C2 both phase separated within 24 hours. Inventive composition A and comparative compositions C3 and C4 remained emulsified and did not phase separate.

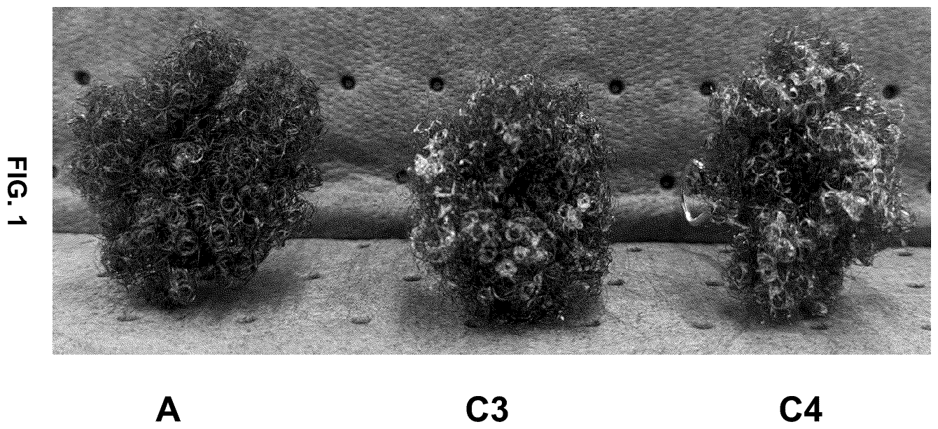

The stable compositions were subsequently used for performance testing. Equal amounts of the stable compositions (inventive composition A and comparative compositions C3 and C4) we separately applied to curl pattern 7 natural hair swatches (0.6 grams of composition were applied to 1.2 grams hair swatches). Each composition was uniformly massaged into the hair swatches, and the hair swatches were allowed to dry naturally under ambient conditions (about 25° C.). After about 24 hours, the hair swatches had completely dried and were visually evaluated. The hair swatches treated with comparative compositions C3 and C4 had formed a very noticeable whitish, flaky residue. The hair swatches treated with inventive composition A, however, showed minimal or no whitish, flaky residue but instead had a natural appearance. Pictures of the dried hair swatches are provided in FIG. 1.

### Example 3

### (In Vivo Performance Evaluation)

Inventive composition A, also shown in Table 1A of Example 1, was evaluated for in vivo performance. To determine whether benefits provided by the inventive composition were at least equivalent to a popular commercial benchmark product, a side-by-side comparison was carried out. Inventive composition A and the commercial benchmark product are set forth in Table 3, below.

TABLE 3

| | | | A | Benchmark |
|---|---|---|---|---|
| (a) | Cationic | BRASSICAMIDOPROPYL | 1.3 | |
| | Surfactant | DIMETHYLAMINE | | |
| | | CETRIMONIUM | | X¹ |
| | | CHLORIDE AND | | |
| | | BEHENTRIMONIUM | | |
| | | CHLORIDE | | |
| (b) | Solid Fatty | MYRISTYL ALCOHOL, | 6.1 | X |
| | Acid | CETEARYL ALCOHOL, | | |
| | | CETYL ALCOHOL, | | |
| Total (a) + (b) | | | 7.4 | X |
| (c) | Liquid FA | LAURYL ALCOHOL | 1.9 | |
| (d) | Low Polar Oil | DICAPRYLYL ETHER | 2.7 | |
| | Silicone Oil | AMODIMETHICONE AND | | X |
| | | DIMETHICONE | | |
| Total (c) + (d) | | | 4.6 | X |
| Ratio ((c) + (d)):((a) + (b)) | | | 0.6:1 | NA |
| (f) | Water Soluble | CAPRYLYL GLYCOL, | 0.3 | X |
| | Solvent | PROPYLENE GLYCOL | | |
| | | GLYCERIN, AND/OR | | |
| | | ISOPROPYL ALCOHOL | | |
| (g) | Organic Acid | TARTARIC ACID | 0.2 | |
| | Nonpolar Oil | Natural Plant Oil | | X |

| | | |
|---|---|---|
| Miscellaneous ² | ≤5 | X |

| | | | |
|---|---|---|---|
| (e) | Water | WATER | Q.S. |

| | | |
|---|---|---|
| Stable | Y | Y |
| Lack or Residue and Flaking | Y | N |

¹An “X” designates the presence of the ingredient.
² Preservatives, fragrances, pH adjusters, salts, chelating agents, buffers, antioxidants, flavonoids, vitamins, botanical extracts, emollients, UV filtering agents, proteins, protein hydrolysates, protein isolates, fillers, composition colorants, or mixtures thereof.

The commercial benchmark product is similar to the compositions of the instant disclosure except it lacks a liquid fatty alcohol. Also, it includes silicone oils (amodimethicone and dimethicone) instead of a non-silicone, low polar oil.

Inventive composition A and the commercial benchmark product were used on the head of hair of a three volunteers. Half of the head of hair of each volunteer was treated with Inventive composition A and the other half of the head of hair of each volunteer was treated with the commercial benchmark product.

Before shampooing or otherwise treating the hair, Inventive composition A and the commercial benchmark product were used as pre-shampoo detangling products. Inventive composition A and the commercial benchmark product were each applied to separate sides of the head of damp hair, massaged into the hair, and evaluated. The entire head of hair was subsequently cleansed with a standard shampoo and rinsed with water, then followed with treatment with a standard conditioner and rinsed with water. After rinsing the conditioner from the hair with water, inventive composition A and the commercial benchmark product were again used as a detangling product, i.e., a post-shampoo detangling product. Inventive composition A and the commercial benchmark product were each applied again to separate sides of the head of hair (the same side as previously treated with each product), massaged into the hair, and evaluated. The entire head of hair was allowed to dry under a rollerball hair dryer, without first rinsing or washing the post-shampoo detangling products from the hair. After about 1 hour, the hair was dry and again evaluated.

The volunteer was instructed not to wash, rinse, or otherwise treat the hair for two days. The volunteer returned after two days for subsequent evaluations.

When the volunteer returned, the hair was evaluated, then the process described above was repeated. Specifically, inventive composition A and the commercial benchmark product were used as pre-shampoo detangling products. Inventive composition A and the commercial benchmark product were each applied to separate sides of the head of damp hair (the same side as previously treated with each product), massaged into the hair, and evaluated. The hair was subsequently cleansed with a standard shampoo and rinsed with water, then treated with a standard conditioner, and rinsed with water. After rinsing the conditioner from the hair with water, inventive composition A and the commercial benchmark product were again used as detangling products, i.e., as post-shampoo detangling products. Inventive composition A and the commercial benchmark product were each applied to separate sides of the head of hair (the same side previously treated with each product), massaged into the hair, and evaluated. The entire head of hair was allowed to dry under a rollerball hair dryer, without first rinsing or washing the post-shampoo detangling products from the hair. After about 1 hour, the hair was dry and again evaluated.

Each evaluation compared the half head of hair treated with inventive composition A with the half head of hair treated with the commercial benchmark product. The half head of hair treated with inventive composition A was evaluated for a variety of cosmetic properties. Each property was ranked as noticeably worse than treatment with the commercial benchmark product (−−), slightly worse than treatment with the commercial benchmark product (−), the same as treatment with the commercial benchmark product (=), slightly better than treatment with the commercial benchmark product (+), or noticeably better than treatment with the commercial benchmark product (++). The results are shown in Table 4 below.
−− Noticeably Worse
− Slightly Worse
=Equal
+ Slightly Better
++ Noticeably Better

TABLE 4

| | Day 1 | Day 3 (Return) |
|---|---|---|
| Moisture/Hydration | NA | + |
| Smoothness | NA | + |
| Shine | NA | + |
| Residue/Flake | NA | + |
| Curl Definition | NA | + |
| Frizz Control | NA | + |
| Sealed End | NA | + |

Pre-Shampoo

| | | |
|---|---|---|
| Detangling | ++ | ++ |
| Passing Finger Ease | ++ | ++ |
| Less Breakage | = | + |
| Slip | + | + |

Post-Shampoo

| | | |
|---|---|---|
| Detangling | ++ | ++ |
| Passing Finger Ease | ++ | ++ |
| Breakage | = | = |
| Moisture/Hydration | ++ | + |
| Smoothness | + | + |

Dry Result

| | | |
|---|---|---|
| Moisture/Hydration | ++ | ++ |
| Smoothness | + | ++ |
| Shine | ++ | ++ |
| Lack of Whitish Residue and | ++ | ++ |
| Flaking | | |
| Curl Definition | = | + |
| Frizz Control | + | + |
| Sealed Ends | + | + |

The data show that inventive composition A performed better or significantly better than the commercial benchmark product for all characteristics evaluated. There were no differences in breakage during pre-shampoo detangling and during post-shampoo detangling on day 1. There was also no difference in less breakage during post-shampoo detangling on day 3, although the commercial benchmark product showed slightly more breakage during pre-shampoo detangling on day 3.

As a pre-shampoo detangling composition, inventive composition A performed noticeably better with regards to detangling and passing finger ease on both day 1 and day 3. Inventive composition A also provided better slip than the commercial benchmark product on both day 1 and day 3.

As a post-shampoo detangling composition, hair treated with inventive composition A performed noticeably better with regards to detangling and passing finger ease on both day 1 and day 3. Hair treated with inventive composition A also exhibited noticeably better moisturization/hydration than hair treated with the commercial benchmark product on day 1 when used as a post-shampoo detangling product. On day 3, hair treated with inventive composition A showed slightly better moisturization/hydration than hair treated with the commercial benchmark product. With respect to smoothness, hair treated with inventive composition A ranked slightly better than the commercial benchmark on both day 1 and day 3.

The evaluations for dry hair and the results are particularly important because consumers' hair is predominately in a dry state throughout the day, especially when consumers are interfacing with other people. Therefore, it is necessary that the hair in the dry state look presentable and exhibit desirable cosmetic properties. In the dry state, hair treated with inventive composition A was better than hair treated with the commercial benchmark product in all aspects, except curl definition on day 1. Inventive composition and the commercial benchmark product provided the same degree of curl definition on day 1. Moisturization/hydration and shine were noticeably better for hair treated with inventive composition A than hair treated with the commercial benchmark product on both day 1 and day 3. Smoothness was also noticeably better for hair treated with inventive composition A than hair treated with the commercial benchmark product on day 3, and slightly better than the commercial benchmark on day 1. Frizz control and sealed ends were both slightly better for hair treated with inventive composition A compared to hair treated with the commercial benchmark product on both days 1 and 3.

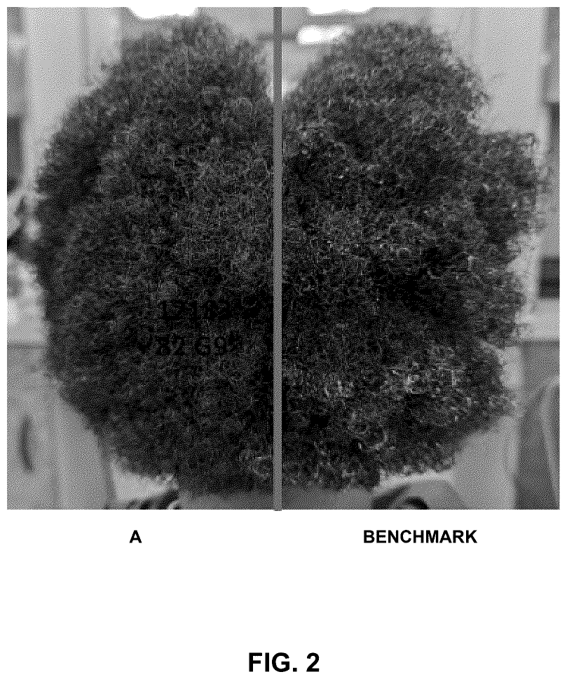

One of the most significant and important findings, however, is with respect to the lack of whitish residue and flaking, as shown in FIG. 2. Hair treated with inventive composition A showed noticeably less (or no) whitish residue and flaking than hair treated with the commercial benchmark product on both day 1 and day 3. Products forming a whitish residue and flaking are particularly objectionable to consumers because the product becomes visible on the hair (having whitish and waxy appearance), and the flaking looks like dandruff. FIG. 2 shows the left head of hair treated with inventive composition A and the right head of hair treated with the commercial benchmark product. The whitish residue is visible.

The inventors not only succeeded in formulating an inventive composition that is at least on par with the popular commercial benchmark product, but also discovered a composition that performs much better than the commercial benchmark product in several criteria. This is significant and surprising, especially considering inventive composition A does not include silicones, which are known for their detangling ability, shine enhancing properties, and ability to impart smoothness to hair. On the other hand, hydrocarbon based fatty compounds are known to be more likely to produce a whitish residue and flaking than silicones. In fact, silicones are often used with hydrocarbon based fatty compounds to prevent formation of whitish residue and flaking. Despite having a higher amount of solid fatty alcohol (compounds that can contribute to formation of whitish residue and flaking) and despite lacking silicones, hair treated with inventive composition A exhibited significantly less (or no) whitish residue and flaking.

Hair treated with inventive composition A was also noticeably easier to detangle, noticeably more moisturized/hydrated, and exhibited significantly better shine.

The differences between the results achieved with inventive composition A and the commercial benchmark product are not due to different cationic surfactants. Inventive composition A includes a dialkylamine (brassicamidopropyl dimethylamine). The commercial benchmark product includes quaternary ammonium compounds (cetrimonium chloride and behentrimonium chloride). Cationic surfactants provide conditioning benefits to hair due to their cationic charge, which attracts the surfactants to the surface of the hair. Quaternary ammonium compounds have a cationic charge due to the quaternary ammonium group. Dialkylamine, however, are “cationizable,” i.e., they can exhibit a cationic charge under acidic conditions.

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number.

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both a fatty compound and a nonionic surfactant. If a particular composition includes both a nonionic surfactant and a fatty compound, a single compound will serve as only the nonionic surfactant or only as the fatty compound (the single compound does not simultaneously serve as both the nonionic surfactant and the fatty component).

A “rinse-off” product refers to a composition that is rinsed and/or washed from the hair with water either after or during the application of the composition onto the hair, and before drying and/or styling the hair. At least a portion of the composition is removed from the hair during the rinsing and/or washing.

A “leave-on” product refers to a composition that is not rinsed and/or washed from the hair after or during application of the composition onto the hair. The composition remains on the hair during drying and/or throughout styling.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair treatment composition comprising:
(a) about 0.1 to about 5 wt. % of one or more cationic surfactants;
(b) about 1 to about 10 wt. % of one or more solid fatty alcohols;
(c) about 0.1 to about 6 wt. % of one or more liquid fatty alcohols;
(d) about 0.3 to about 8 wt. % of one or more low polar oils having a Polarity Index of about 23 mN/m to about 40 mN/m;
wherein a weight ratio of a combination of (c) and (d) to a combination of (a) and (b) is from about 0.5:1 to about 3:1 (((c)+(d)):((a)+(b))); and
(e) about 60 to about 92 wt. % of water;
wherein all percentages by weight are based on a total weight of the composition.

2. The hair treatment composition of claim 1, wherein the one or more cationic surfactants are selected from quaternary ammonium compounds, fatty dialkylamines, fatty amidoamines, salts thereof, or mixtures thereof.

3. The hair treatment composition of claim 1, wherein the one or more cationic surfactants are selected from cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, salts thereof, or mixtures thereof.

4. The hair treatment composition of claim 1, wherein the one or more solid fatty alcohols are selected from stearyl alcohol, cetyl alcohol, cetearyl alcohol, myristyl alcohol, behenyl alcohol, or mixtures thereof.

5. The hair treatment composition of claim 1, wherein (a) and (b) are in a combined amount of about 2 to about 15 wt. %.

6. The hair treatment composition of claim 1, wherein the one or more liquid fatty alcohols are selected from isostearyl alcohol, oleyl alcohol, octyldodecanol, linoleyl alcohol, linolenyl alcohol, lauryl alcohol, 2-hexyldecanol, 2-heptyldecanol, enanthic alcohol, caprylic alcohol, pelargonic alcohol, capric alcohol, or mixtures thereof.

7. The hair treatment composition of claim 1, wherein the one or more low polar oils are selected from fatty carbonates, fatty ethers, liquid linear or branched carboxylic acid esters, or mixtures thereof.

8. The hair treatment composition of claim 1, wherein (c) and (d) are in a combined amount of about 2 to about 15 wt. %.

9. The hair treatment composition of claim 1, further comprising:
(f) about 0.05 to about 15 wt. % of one or more water soluble solvents selected from glycerin, polyols, glycols, or mixtures thereof.

10. The hair treatment composition of claim 1, further comprising one or more fatty compounds other than (b), (c), and (d).

11. The hair treatment composition of claim 1, further comprising one or more cationic conditioning polymers.

12. The hair treatment composition of claim 1, wherein the composition is an oil-in-water emulsion.

13. A hair treatment composition comprising:
(a) one or more cationic surfactants;
(b) one or more solid fatty alcohols;
wherein (a) and (b) are in a combined amount of about 2 to about 15 wt. %;
(c) one or more liquid fatty alcohols;
(d) one or more low polar oils having a Polarity Index of about 23 mN/m to about 40 mN/m;
wherein (c) and (d) are in a combined amount of about 2 to about 15 wt. %;
and a weight ratio of a combination of (c) and (d) to a combination of (a) and (b) is from about 0.5:1 to about 3:1 (((c)+(d)):((a)+(b))); and
(e) water;
wherein all percentages by weight are based on a total weight of the composition.

14. The hair treatment composition of claim 13, wherein the one or more cationic surfactants are in an amount of about 0.5 to about 5 wt. %.

15. The hair treatment composition of claim 13, wherein the one or more solid fatty alcohols are in an amount of about 1 to about 10 wt. %.

16. The hair treatment composition of claim 13, wherein the one or more liquid fatty alcohols are in an amount of about 0.3 to about 6 wt. %.

17. The hair treatment composition of claim 13, wherein the one or more low polarity oils are in an amount of about 0.3 to about 6 wt. %.

18. A hair treatment composition comprising:
(a) about 0.5 to about 3 wt. % of one or more cationic surfactants;
(b) about 3 to about 10 wt. % of one or more solid fatty alcohols selected from stearyl alcohol, cetyl alcohol, cetearyl alcohol, myristyl alcohol, behenyl alcohol, arachidyl alcohol, or mixtures thereof;
wherein (a) and (b) are in a combined amount of about 2 to about 15 wt. %;
(c) about 0.5 to about 4 wt. % of one or more liquid fatty alcohols selected from isostearyl alcohol, lauryl alcohol, oleyl alcohol, octyldodecanol, and mixtures thereof;
(d) about 1 to about 5 wt. % of one or more low polar oils having a Polarity Index of about 23 mN/m to about 40 mN/m selected from fatty carbonates, fatty ethers, liquid linear or branched carboxylic acid esters, or mixtures thereof;
wherein (c) and (d) are in a combined amount of about 2 to about 15 wt. %; and
wherein (a), (b), (c), and (d) are in a weight ratio of (c) and (d) to (a) and (b) of about 0.5:1 to about 2:1 (((c)+(d)):((a)+(b)));
(e) about 75 to about 90 wt. % of water; and
(f) optionally, about 0.1 to about 10 wt. % of one or more miscellaneous ingredients;
wherein all percentages by weight are based on a total weight of the composition.

19. A method for treating hair comprising applying the hair treatment composition of claim 1 to the hair.

20. The method of claim 19, further comprising detangling the hair while the hair treatment composition is applied to the hair.
